(19) United States
(12) Patent Application Publication
(10) Pub. No.: US 2025/0186417 A1
(43) Pub. Date: Jun. 12, 2025

(54) CYTISINE FORMULATIONS

(71) Applicant: Achieve Life Sciences, Inc., Bothell, WA (US)

(72) Inventors: Marco Antonio Delgado Rueda, Bucks (GB); Kelly Miller, Bothell, WA (US); Craig Donnelly, Bothell, WA (US); David Elder, Stevenage (GB); Andrew Searle, Stevenage (GB)

(21) Appl. No.: 18/977,247

(22) Filed: Dec. 11, 2024

Related U.S. Application Data
(60) Provisional application No. 63/608,959, filed on Dec. 12, 2023.

(51) Int. Cl. A61K 31/439; A61K 9/00; A61K 9/20; A61K 31/198; A61K 33/00

(52) U.S. Cl. CPC A61K 31/439; A61K 9/0053; A61K 9/20; A61K 31/198; A61K 33/00

(57) ABSTRACT

Provided herein is a cytisine formulation comprising cytisine, a nitrite scavenger, a glidant, a lubricant, a buffering agent and one or more fillers, wherein, upon storage of the composition at about 60° C., the amount of N-nitrosocytisine relative to a total weight of cytisine is 4.2 ng/mg or less after about 30 days.

### CROSS-REFERENCE TO RELATED APPLICATIONS

This application claims the benefit of U.S. Provisional Patent Application 63/608,959 filed Dec. 12, 2023, which is incorporated herein by reference in its entirety.

### BACKGROUND

Nicotine is an addictive substance that is rapidly absorbed during cigarette smoking which contributes to some 7 million premature deaths each year worldwide. Smoking is highly addictive, with more than 95% of unaided attempts at cessation failing to last 6 months. It has been estimated that for every year that a person continues smoking beyond his or her mid-30s, that person loses 3 months of life expectancy.

The pharmacotherapies currently available in the U.S. and Western Europe to help smokers stop include nicotine replacement therapy (NRT) and two non-nicotine containing medications: bupropion (Zyban®, Glaxo-SmithKline) and varenicline (Chantix®/Champix®, Pfizer). NRT and bupropion appear to have about equal efficacy. Varenicline is more effective than single NRT and bupropion, although combination NRT is comparable in efficacy. Tabex®, containing the active substance (−)-cytisine (cytisinicline; commonly referred to simply as cytisine), has been licensed and marketed in Central and Eastern Europe for several decades by Sopharma PLC (Sophia, Bulgaria).

N-nitrosamine compounds, which contain nitroso groups having the chemical structure N(R)(R¹)—N═O), can be formed from secondary, tertiary, or quaternary amines in the presence of nitrous acid (nitrite salts under acidic conditions). Short chain alkyl N-nitrosamine impurities including NDMA N-nitrosodimethylamine (NDMA), N-nitrosodiethylamine (NDEA), N-nitrosomethylethylamine (NEMA), N-nitrosoisopropylethylamine (NIPEA), N nitroso di-isopropylamine (NDIPA), N-nitroso-N-methylaniline (NMA), N-nitrosodi-n-propylamine (NDPA), N-nitrosopiperidine (NPP) and N-nitrosodi-n-butylamine (NDBA), have been identified by the U.S. Food and Drug Administration (FDA) as potentially being present in drug substances or drug products. Further, N-nitrosamine drug substance related impurities (NDSRIs) have been determined to be present in various drug products, such as, for example, varenicline, bupropion, and cytisine related products. Global health agencies such as the FDA and European Medicines Agency (EMA) have requested three-stage risk assessments for potential N-nitrosamine contamination for all commercial medicinal products in their regions and have developed recommended acceptable intake limits for NDSRIs.

Therefore, a need exists for nicotine addiction treatments comprising low levels of NDSRIs.

### SUMMARY

In some aspects, the present technology includes a cytisine formulation comprising cytisine, a nitrite scavenger, a glidant, a lubricant, a buffering agent, and one or more fillers, wherein, upon storage of the formulation at about 60° C., the amount of N-nitrosocytisine relative to a total weight of cytisine, (c) is 4.2 ng/mg or less after about 30 days.

In some embodiments, the cytisine is pretreated with the nitrite scavenger. In some embodiments, the one or more fillers are pretreated with the nitrite scavenger.

In other aspects, provided herein is a cytisine tablet, comprising 1 wt % to 5 wt % cytisine, 1 wt % to 10 wt % L-cysteine, 0.5 wt % to 5 wt % colloidal silicon dioxide, 0.5 wt % to 5 wt % magnesium stearate, 1 wt % to 5 wt % KH₂PO₄, 20 wt % to 35 wt % powdered cellulose, and 50 wt % to 70 wt % calcium sulfate dihydrate, wherein, upon storage of the tablet at about 60° C., the amount of N-nitrosocytisine relative to a total weight of cytisine, (c) is 4.2 ng/mg or less after about 30 days.

In some embodiments, the cytisine is pretreated with the nitrite scavenger. In some embodiments, the powdered cellulose, the calcium sulfate dihydrate, or both are pretreated with the nitrite scavenger.

### BRIEF DESCRIPTION OF THE DRAWINGS

FIGS. 1A-1C are graphs showing the impact of pH on N-nitrosocytisine (NNC) formation in accordance with the present technology.

FIGS. 2A-2C are graphs showing the impact of L-cysteine on NNC formation in accordance with the present technology.

FIGS. 3A and 3B are graphs showing the formation of related impurities in uncoated and coated tablets in accordance with the present technology.

FIG. 4 is a graph showing the contribution of N-formyl cytisine (NFC) to total related impurities in accordance with the present technology.

FIGS. 5A and 5B are graphs showing a comparison of the impact of pH vs L-cysteine on NNC formation in uncoated and coated tablets under air in accordance with the present technology.

FIGS. 6A-6E are graphs showing the impact of L-cysteine loading on NNC formation in uncoated and coated tablets under air in accordance with the present technology.

FIGS. 7A and 7B are graphs showing the impact of L-cysteine loading on NNC formation in uncoated and coated tablets under air in accordance with the present technology.

FIGS. 8A and 8B are graphs showing the impact of blend ageing with L-cysteine on NNC formation in uncoated and coated tablets under air in accordance with the present technology.

### DETAILED DESCRIPTION

As a cyclic secondary amine, cytisine is a nitrosatable compound. As shown below, under acidic conditions, cytisine can be converted to N-nitrosocytisine (NNC) in the presence of residual nitrites:

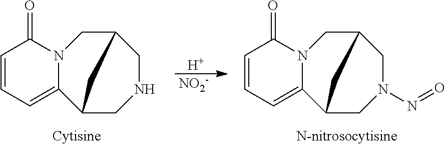

As such, compositions comprising cytisine can be associated with low levels of N-nitrosamine impurities. Further, the level of such impurities may increase when cytisine is stored for an extended period of time, e.g., 30 days, 60 days, 120 days, 6 months, 12 months, 18 months, or 24 months. The presence of N-nitrosamine impurities such as alkyl N-nitrosoamines, e.g., NDMA, NDEA, NEMA, NIPEA, NDIPA, NMA, NDPA, NPP, and NDBA; and NDSRIs, e.g., N-nitrosocytisine in compositions comprising cytisine may render such compositions unsafe for consumption.

The present technology relates to cytisine formulations (e.g., pharmaceutical compositions and dosage units) wherein the amount of N-nitrosamine impurities is controlled and/or reduced relative to the amount of N-nitrosamine impurities present in conventional cytisine formulations. In particular, the N-nitrosamine impurities may comprise N-nitrosocytisine (NCC). Cytisine formulations described herein may have an extended shelf-life compared to the shelf-life of conventional cytisine formulations.

While the present disclosure is capable of being embodied in various forms, the description below of several embodiments is made with the understanding that the present disclosure is to be considered as an exemplification of the present technology and is not intended to limit the present technology to the specific embodiments illustrated. Headings are provided for convenience only and are not to be construed to limit the present technology in any manner. Embodiments illustrated under any heading may be combined with embodiments illustrated under any other heading.

### Definitions

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

The disclosure of ranges is intended as a continuous range, including every value between the minimum and maximum values recited, as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and, in all instances, such ratios, ranges, and ranges of ratios represent various embodiments of the present disclosure.

As used herein, the terms “low N-nitrosocytisine content” and “low NNC content” refer to an amount of NNC, relative to a total weight of cytisine, of less than about 50 ng/mg, less than about 45 ng/mg, less than about 40 ng/mg, less than about 35 ng/mg, less than about 30 ng/mg, less than about 25 ng/mg, less than about 20 ng/mg, less than about 15 ng/mg, or less than about 10 ng/mg. In some embodiments, a cytisine formulation comprising low NNC content comprises an amount of NNC, relative to a total weight of cytisine, of less than about 5 ng/mg, less than about 4.2 ng/mg, less than about 3 ng/mg, less than about 2 ng/mg, or less than about 1.5 ng/mg. Cytisine formulations comprising low NNC content may comprise an amount of NNC within the daily acceptable intake limit set by a health agency. In some embodiments, cytisine formulations comprising low NNC content comprise an amount of NNC within a daily acceptable limit including, but not limited to, about 600 ng/day or less, about 550 ng/day or less, about 500 ng/day or less, about 450 ng/day or less, about 400 ng/day or less, about 350 ng/day or less, about 300 ng/day or less, about 250 ng/day or less, about 200 ng/day or less, about 150 ng/day or less, about 100 ng/day or less, about 90 ng/day or less, about 80 ng/day or less, about 70 ng/day or less, about 60 ng/day or less, about 50 ng/day or less, about 40 ng/day or less, about 30 ng/day or less, about 20 ng/day or less, about 10 ng/day or less, or about 5 ng/day or less.

The term “low nitrite content” may be used in reference to the nitrite content of one or more pharmaceutically acceptable excipients and means a nitrite content of less than about 50 parts per million (ppm). In some embodiments, one or more pharmaceutically acceptable excipients comprise a nitrite content of less than about 50 ppm, less than about 25 ppm, less than about 10 ppm, or less than about 5 ppm. In some embodiments, the one or more pharmaceutically acceptable excipients comprise a nitrite content, relative to the total weight of the pharmaceutical composition of about 1 ppm to about 10 ppm. In some embodiments, one or more pharmaceutically acceptable excipients comprise a nitrite content of about 1.2 ppm, about 1 ppm, about 0.8 ppm, about 0.6 ppm, about 0.4 ppm, about 0.2 ppm, or about 0.1 ppm. Certain pharmaceutically acceptable excipients, such as, e.g., fillers and buffering agents, may have low nitrite content. Certain other pharmaceutically acceptable excipients, such as, e.g., diluents and glidants, may have a standard nitrite content.

“Comprising” or “comprises” is intended to mean that the formulations include the recited elements, but not excluding others. “Consisting essentially of” when used to define formulations, shall mean excluding other elements of any essential significance to the combination for the stated purpose. Thus, a cytisine formulation consisting essentially of the elements as defined herein would not exclude other materials or steps that do not materially affect the basic and novel characteristic(s) of the claimed present technology. “Consisting of” shall mean excluding more than trace elements of other ingredients. Embodiments defined by each of these transition terms are within the scope of this present technology.

As used herein, the term “drug substance” refers to a compound (e.g., cytisine) that is biologically active, e.g., an active pharmaceutical ingredient (API). The term “drug product” refers to a composition that contains a drug substance (i.e., active ingredient) and optionally a pharmaceutically acceptable carrier and/or excipient.

The term “pharmaceutically acceptable” in the present context means that the substance in question does not produce unacceptable toxicity to the subject or interaction with other components of the composition.

The terms “orally deliverable” or “oral administration” herein include any form of delivery of a therapeutic agent or a composition thereof to a subject wherein the agent or composition is placed in the mouth of the subject, whether or not the agent or composition is swallowed. Thus, “oral administration” includes buccal and sublingual, as well as esophageal administration.

### Cytisine Formulations

In one aspect, the present technology relates to a cytisine formulation, wherein the formulation has a low level of N-nitrosamine impurities. In some embodiments, the cytisine formulation has a low level of N-nitrosocytisine (NNC).

In some embodiments, the cytisine formulation has an amount of NNC, relative to a total weight of cytisine, of less than about 50 ng/mg, less than about 45 ng/mg, less than about 40 ng/mg, less than about 35 ng/mg, less than about 30 ng/mg, less than about 25 ng/mg, less than about 20 ng/mg, less than about 15 ng/mg, or less than about 10 ng/mg. In some embodiments, the cytisine formulation has an amount of NNC, relative to a total weight of cytisine, of less than about 10 ng/mg, less than about 5 ng/mg, less than about 4 ng/mg, less than about 3 ng/mg, or less than about 2 ng/mg.

As will be appreciated by one of ordinary skill in the art, the amount of NDSRIs in a drug substance or drug product (e.g., a cytisine formulation) may increase upon storage for a period of time. Thus, the present technology may control and/or reduce the amount of NNC in a cytisine formulation upon storage at 25° C./60% relative humidity (RH) and 40° C./75% RH or other conditions such as 60° C. For example, a cytisine formulation in accordance with the present technology, upon storage of the composition at 25° C./60% RH and 40° C./75% RH or other conditions such as 60° C. for a length of time, may comprise a lower level of NNC than a conventional cytisine formulation stored under substantially similar conditions. In some embodiments, upon storage of the cytisine formulation at about 60° C., the amount of NNC is about 4.2 ng/mg or less after about 30 days. In some embodiments, upon storage of the cytisine formulation at about 60° C., the amount of NNC is about 4 ng/mg or less after about 30 days. In some embodiments, upon storage of the cytisine formulation at about 60° C., the amount of a NNC is about 3 ng/mg or less after about 30 days. In some embodiments, upon storage of the cytisine formulation at about 60° C., the amount of NNC is about 45 ng/mg or less after about 30 days. In some embodiments, upon storage of the cytisine formulation at about 60° C., the amount of NNC is about 30 ng/mg or less after about 30 days.

In some embodiments, upon storage of the cytisine formulation at ambient conditions (i.e., 25° C./60% RH), the amount of NNC is about 4 ng/mg or less after about 18 months. In some embodiments, upon storage of the cytisine formulation at ambient conditions, the amount of NNC is about 4 ng/mg or less after about 24 months or after about 36 months. In some embodiments, upon storage of the cytisine formulation at ambient conditions (i.e., 25° C./60% RH), the amount of NNC is about 4.2 ng/mg or less after about 18 months. In some embodiments, upon storage of the cytisine formulation at ambient conditions, the amount of NNC is about 4.2 ng/mg or less after about 24 months or after about 36 months. In other embodiments, upon storage of the cytisine formulation at ambient conditions, the amount of NNC is about 45 ng/mg or less after about 18 months. In some embodiments, upon storage of the cytisine formulation at ambient conditions, the amount of NNC is about 45 ng/mg or less after about 24 months or after about 36 months.

Cytisine may be present in the cytisine formulation in an amount sufficient to achieve the desired treatment effect, e.g., smoking cessation. In some embodiments, cytisine is present in an amount of about 0.5 wt % to about 10 wt %, relative a total weight of the cytisine formulation. In some embodiments, cytisine is present in an amount of about 1 wt % to about 10 wt %, relative the total weight of the cytisine formulation. In some embodiments, cytisine is present in an amount of about 0.5 wt % to about 5 wt %, relative the total weight of the cytisine formulation. In some embodiments, cytisine is present in an amount of about 1 wt % to about 5 wt %, relative the total weight of the cytisine formulation. In some embodiments, cytisine is present in an amount of about 1.5 wt % to about 5 wt %, relative the total weight of the cytisine formulation. In some embodiments, cytisine is present in an amount of about 1 wt % to about 3 wt %, relative the total weight of the cytisine formulation. In some embodiments, cytisine is present in an amount of about 1.5 wt % to about 3 wt %, relative the total weight of the cytisine formulation.

In order to control and/or reduce the amount of N-nitrosamine impurities, the cytisine formulation comprises a nitrite scavenger. Any compound known to scavenge nitrites, block nitrosation reactions, and ultimately prevent or significantly reduce the formation of nitrosamines may be used as a nitrite scavenger in the present technology. In some embodiments, the nitrite scavenger is one or more amino acids, reducing agents, and antioxidants. Non-limiting examples of nitrite scavengers include amino acids such as homocysteine, L-cysteine, D-cysteine, glycine, alanine, methionine, lysine, taurine, glutathione, cystine, amino sulfamic acids, and amino sulfonic acids; reducing agents such as ascorbic acid; and antioxidants such as vitamin A, vitamin E, propyl gallate, glutathione, and butylated hydroxytoluene (BHT). In some embodiments, the nitrite scavenger is L-cysteine. In some embodiments, when the nitrite scavenger is L-cysteine, the L-cysteine is in the form of a salt, such as, e.g., a hydrochloride. In such embodiments, the L-cysteine may be L-cysteine hydrochloride or L-cysteine hydrochloride anhydrous. Accordingly, in some embodiments, the L-cysteine is L-cysteine hydrochloride. In other embodiments, the L-cysteine is L-cysteine hydrochloride anhydrous. In yet other embodiments, the L-cysteine is free base L-cysteine.

In some embodiments of the cytisine formulation, the nitrite scavenger is present in the cytisine formulation in an amount sufficient to control and/or reduce the amount of N-nitrosamine impurities, e.g., NNC, in the formulation. In some embodiments, the nitrite scavenger is present in the cytisine formulation in an amount of about 1 wt % to about 10 wt %, relative to a total weight of the formulation. In some embodiments, the nitrite scavenger is present in the cytisine formulation in an amount of about 1 wt % to about 7 wt %, relative to the total weight of the formulation. In some embodiments, the nitrite scavenger is present in the cytisine formulation in an amount of about 3 wt % to about 10 wt %, relative to the total weight of the formulation. In some embodiments, the nitrite scavenger is present in the cytisine formulation in an amount of about 3 wt % to about 7 wt %, relative to the total weight of the formulation. In some embodiments, the nitrite scavenger is present in the cytisine formulation in an amount of about 1 wt % to about 5 wt %, relative to the total weight of the formulation. In some embodiments, the nitrite scavenger is present in the cytisine formulation in an amount of about 3 wt % to about 5 wt %, relative to the total weight of the formulation. In some embodiments, the nitrite scavenger is present in the cytisine formulation in an amount of about 5 wt % to about 7 wt %, relative to the total weight of the formulation. In some embodiments, the nitrite scavenger is present in the cytisine formulation in an amount of about 5 wt %, relative to the total weight of the formulation. In some embodiments, the nitrite scavenger is present in the cytisine formulation in an amount of about 3 wt %, relative to the total weight of the formulation. In some embodiments, the nitrite scavenger is present in the cytisine formulation in an amount of about 5 wt %, relative to the total weight of the formulation. In some embodiments, the nitrite scavenger is present in the cytisine formulation in an amount of about 7 wt %, relative to the total weight of the formulation.

In some embodiments of the cytisine formulation, the nitrite scavenger is L-cysteine (in any designated form as the free base equivalent) and is present in the formulation in an amount sufficient to control and/or reduce the amount of N-nitrosamine impurities, e.g., NNC, in the formulation. In some embodiments, L-cysteine is present in the cytisine formulation in an amount of about 1 wt % to about 10 wt %, relative to a total weight of the formulation. In some embodiments, L-cysteine is present in the cytisine formulation in an amount of about 1 wt % to about 7 wt %, relative to the total weight of the formulation. In some embodiments, L-cysteine is present in the cytisine formulation in an amount of about 3 wt % to about 10 wt %, relative to the total weight of the formulation. In some embodiments, L-cysteine is present in the cytisine formulation in an amount of about 3 wt % to about 7 wt %, relative to the total weight of the formulation. In some embodiments, L-cysteine is present in the cytisine formulation in an amount of about 1 wt % to about 5 wt %, relative to the total weight of the formulation. In some embodiments, L-cysteine is present in the cytisine formulation in an amount of about 3 wt % to about 5 wt %, relative to the total weight of the formulation. In some embodiments, L-cysteine is present in the cytisine formulation in an amount of about 5 wt % to about 7 wt %, relative to the total weight of the formulation. In some embodiments, L-cysteine is present in the cytisine formulation in an amount of about 5 wt %, relative to the total weight of the formulation. In some embodiments, L-cysteine is present in the cytisine formulation in an amount of about 3 wt %, relative to the total weight of the formulation. In some embodiments, L-cysteine is present in the cytisine formulation in an amount of about 5 wt %, relative to the total weight of the formulation. In some embodiments, L-cysteine is present in the cytisine formulation in an amount of about 7 wt %, relative to the total weight of the formulation.

Cytisine formulations in accordance with the present technology may further comprise one or more pharmaceutically acceptable excipients, such as those common in the art. Excipients that may be included in the formulations include, for example, fillers, disintegrants, glidants, lubricants, wetting agents, sweetening agents, flavoring agents, and coloring agents. Such pharmaceutically acceptable excipients may be employed in the formulations to improve processability, palatability, stability, and bioavailability of the formulation. Suitable pharmaceutically acceptable excipients that may be included in the cytisine formulations of the present technology include, but are not limited to, corn starch, rice starch, potato starch, starch 1500, pregelatinized starch, partially pregelatinized starch, microcrystalline cellulose, microfine cellulose, powdered cellulose, ethyl cellulose, glyceryl behenate, sodium starch glycolate, soy polysaccharide (Emcosoy), calcium sulfate, dicalcium phosphate, dibasic calcium phosphate, tricalcium phosphate, calcium carbonate, calcium stearate, zinc stearate, calcium silicate, magnesium stearate, magnesium lauryl sulfate, magnesium carbonate, magnesium oxide, talc, stearic acid, colloidal silicon dioxide, fumed silica (Cab-O-Sil), dextrates, sucrose, dextrose, crospovidone, sorbitol, mannitol, maltrodextrine, and glucose monohydrate.

In some embodiments, the cytisine formulation includes one or more pharmaceutically acceptable excipients selected from powdered cellulose, calcium sulfate dihydrate, colloidal silicon dioxide, and magnesium stearate. In some embodiments, the one or more pharmaceutically acceptable excipients comprise powdered cellulose. In some embodiments, the one or more pharmaceutically acceptable excipients comprise colloidal silicon dioxide. In some embodiments, the one or more pharmaceutically acceptable excipients comprise calcium sulfate dihydrate. In some embodiments, the one or more pharmaceutically acceptable excipients comprise magnesium stearate. In some embodiments, the one or more pharmaceutically acceptable excipients comprise powdered cellulose, colloidal silicon dioxide, calcium sulfate dihydrate, and magnesium stearate.

Some commonly used pharmaceutically acceptable excipients, such as, e.g., powdered cellulose, colloidal silicon dioxide, calcium sulfate dihydrate, and magnesium stearate, may naturally have trace amounts of nitrites. As such, the one or more pharmaceutically acceptable excipients included in cytisine formulations in accordance with the present technology may be pretreated with a nitrite scavenger. In some embodiments, all of the one or more pharmaceutically acceptable excipients are pretreated with a nitrite scavenger. In some embodiments, only some, e.g., one or two, of the pharmaceutically acceptable excipients are pretreated with a nitrite scavenger. In particular, pharmaceutically acceptable excipients that are present in the cytisine formulation in an amount greater than or equal to about 30 wt %, about 40 wt %, about 50 wt %, or about 60 wt %, relative to a total weight of the formulation, may be pretreated with a nitrite scavenger. On the other hand, pharmaceutically acceptable excipients that are present in the cytisine formulation in an amount less than about 20 wt %, less than about 15 wt %, less than about 10 wt %, less than about 5 wt %, or less than about 2.5 wt %, relative to the total weight of the formulation, may not be pretreated with a nitrite scavenger.

Accordingly, in some embodiments of the cytisine formulation, the one or more pharmaceutically acceptable excipients comprise a nitrite content, relative to a total weight of the formulation, of less than about 50 ppm, less than about 25 ppm, less than about 10 ppm, or less than about 5 ppm. In some embodiments, the one or more pharmaceutically acceptable excipients comprise a nitrite content, relative to the total weight of the pharmaceutical composition of about 1 ppm to about 10 ppm. In some embodiments, the one or more pharmaceutically acceptable excipients comprise a nitrite content, relative to the total weight of the pharmaceutical composition of less than 1.2 ppm, less than 1.0 ppm, less than 0.8 ppm, less than 0.6 ppm, less than 0.4 ppm, less than 0.2 ppm, or less than 0.1 ppm.

In some embodiments, the cytisine formulation further comprises one or more pharmaceutically acceptable excipients in an amount ranging from about 70 wt % to about 97.5 wt %, relative to the total weight of the formulation. In some embodiments, the cytisine formulation further comprises one or more pharmaceutically acceptable excipients in an amount ranging from about 75 wt % to about 95 wt %, relative to the total weight of the formulation. In some embodiments, the cytisine formulation further comprises one or more pharmaceutically acceptable excipients in an amount ranging from about 80 wt % to about 95 wt %, relative to the total weight of the formulation. In some embodiments, the cytisine formulation further comprises one or more pharmaceutically acceptable excipients in an amount ranging from about 80 wt % to about 90 wt %, relative to the total weight of the formulation. In some embodiments, the cytisine formulation further comprises one or more pharmaceutically acceptable excipients in an amount ranging from about 85 wt % to about 90 wt %, relative to the total weight of the formulation. In some embodiments, the cytisine formulation further comprises one or more pharmaceutically acceptable excipients in an amount of about 89.7 wt %, relative to the total weight of the formulation.

In some embodiments of the cytisine formulation, the one or more pharmaceutically acceptable excipients comprise powdered cellulose, colloidal silicon dioxide, calcium sulfate dihydrate, and magnesium stearate, and the one or more pharmaceutically acceptable excipients are present in the formulation in an amount ranging from about 70 wt % to about 97.5 wt %, relative to a total weight of the formulation. In some embodiments of the cytisine formulation, the one or more pharmaceutically acceptable excipients comprise powdered cellulose, colloidal silicon dioxide, calcium sulfate dihydrate, and magnesium stearate, and the one or more pharmaceutically acceptable excipients are present in the formulation in an amount ranging from about 80 wt % to about 95 wt %. In some embodiments of the cytisine formulation, the one or more pharmaceutically acceptable excipients comprise powdered cellulose, colloidal silicon dioxide, calcium sulfate dihydrate, and magnesium stearate, and the one or more pharmaceutically acceptable excipients are present in the formulation in an amount ranging from about 80 wt % to about 90 wt %. In some embodiments of the cytisine formulation, the one or more pharmaceutically acceptable excipients comprise powdered cellulose, colloidal silicon dioxide, calcium sulfate dihydrate, and magnesium stearate, and the one or more pharmaceutically acceptable excipients are present in the formulation in an amount ranging from about 85 wt % to about 90 wt %. In some embodiments of the cytisine formulation, the one or more pharmaceutically acceptable excipients comprise powdered cellulose, colloidal silicon dioxide, calcium sulfate dihydrate, and magnesium stearate, and the one or more pharmaceutically acceptable excipients are present in the formulation in an amount of about 89.7 wt %.

In some embodiments, the one or more pharmaceutically acceptable excipients comprise powdered cellulose in an amount ranging from about 20 wt % to about 35 wt %, relative to a total weight of the formulation. In some embodiments, the one or more pharmaceutically acceptable excipients comprise powdered cellulose in an amount ranging from about 25 wt % to about 30 wt %, relative to the total weight of the formulation. In some embodiments, the one or more pharmaceutically acceptable excipients comprise powdered cellulose in an amount of about 28.5 wt %, relative to the total weight of the formulation.

In some embodiments, the one or more pharmaceutically acceptable excipients comprise calcium sulfate dihydrate in an amount ranging from about 50 wt % to about 70 wt %, relative to a total weight of the formulation. In some embodiments, the one or more pharmaceutically acceptable excipients comprise calcium sulfate dihydrate in an amount ranging from about 55 wt % to about 65 wt %, relative to the total weight of the formulation. In some embodiments, the one or more pharmaceutically acceptable excipients comprise calcium sulfate dihydrate in an amount ranging from about 55 wt % to about 60 wt %, relative to the total weight of the formulation. In some embodiments, the one or more pharmaceutically acceptable excipients comprise calcium sulfate dihydrate in an amount of about 58.2 wt %, relative to the total weight of the formulation.

In some embodiments, the one or more pharmaceutically acceptable excipients comprise colloidal silicon dioxide in an amount ranging from about 0.5 wt % to about 5 wt %, relative to a total weight of the formulation. In some embodiments, the one or more pharmaceutically acceptable excipients comprise colloidal silicon dioxide in an amount ranging from about 1.0 wt % to about 3.0 wt %, relative to the total weight of the formulation. In some embodiments, the one or more pharmaceutically acceptable excipients comprise colloidal silicon dioxide in an amount ranging from about 1.0 wt % to about 2.0 wt %, relative to the total weight of the formulation. In some embodiments, the one or more pharmaceutically acceptable excipients comprise colloidal silicon dioxide in an amount of about 1.5 wt %, relative to the total weight of the formulation.

In some embodiments, the one or more pharmaceutically acceptable excipients comprise magnesium stearate in an amount ranging from about 0.5 wt % to about 5 wt %, relative to a total weight of the formulation. In some embodiments, the one or more pharmaceutically acceptable excipients comprise magnesium stearate in an amount ranging from about 1.0 wt % to about 3.0 wt %, relative to the total weight of the formulation. In some embodiments, the one or more pharmaceutically acceptable excipients comprise magnesium stearate in an amount ranging from about 1.0 wt % to about 2.0 wt %, relative to the total weight of the formulation. In some embodiments, the one or more pharmaceutically acceptable excipients comprise magnesium stearate in an amount of about 1.5 wt %, relative to the total weight of the formulation.

In some embodiments, the cytisine formulation includes one or more pharmaceutically acceptable excipients selected from lubricants, glidants, and fillers. In some embodiments, the one or more pharmaceutically acceptable excipients comprise a lubricant. In some embodiments, the one or more pharmaceutically acceptable excipients comprise a glidant. In some embodiments, the one or more pharmaceutically acceptable excipients comprise one or more fillers. In some embodiments, the cytisine formulation comprises a lubricant, a glidant, and one or more fillers.

In some embodiments, the cytisine formulation comprises one or more fillers in an amount ranging from about 70 wt % to about 95 wt %, relative to a total weight of the formulation. In some embodiments, the cytisine formulation comprises one or more fillers in an amount ranging from about 75 wt % to about 92.5 wt %, relative to a total weight of the formulation. In some embodiments, the cytisine formulation comprises one or more fillers in an amount ranging from about 80 wt % to about 90 wt %, relative to a total weight of the formulation. In some embodiments, the cytisine formulation comprises one or more fillers in an amount ranging from about 85 wt % to about 87.5 wt %, relative to a total weight of the formulation. In some embodiments, the cytisine formulation comprises one or more fillers in an amount of about 86.7 wt %, relative to a total weight of the formulation.

In some embodiments of the cytisine formulation, the one or more fillers comprise powdered cellulose. In some embodiments the one or more fillers comprise powdered cellulose in an amount ranging from about 20 wt % to about 35 wt %, relative to a total weight of the formulation. In some embodiments, the one or more fillers comprise powdered cellulose in an amount ranging from about 25 wt % to about 30 wt %, relative to the total weight of the formulation. In some embodiments, the one or more fillers comprise powdered cellulose in an amount of about 28.5 wt %, relative to the total weight of the formulation.

In some embodiments of the cytisine formulation, the one or more fillers comprise calcium sulfate dihydrate. In some embodiments, the one or more fillers comprise calcium sulfate dihydrate in an amount ranging from about 50 wt % to about 70 wt %, relative to a total weight of the formulation. In some embodiments, the one or more fillers comprise calcium sulfate dihydrate in an amount ranging from about 55 wt % to about 65 wt %, relative to the total weight of the formulation. In some embodiments, the one or more fillers comprise calcium sulfate dihydrate in an amount ranging from about 55 wt % to about 60 wt %, relative to the total weight of the formulation. In some embodiments, the one or more fillers comprise calcium sulfate dihydrate in an amount of about 58.2 wt %, relative to the total weight of the formulation.

In some embodiments, the cytisine formulation comprises a glidant in an amount ranging from about 0.5 wt % to about 5 wt %, relative to a total weight of the formulation. In some embodiments, the cytisine formulation comprises a glidant in an amount ranging from about 1.0 wt % to about 3.0 wt %, relative to the total weight of the formulation. In some embodiments, the cytisine formulation comprises a glidant in an amount ranging from about 1.0 wt % to about 2.0 wt %, relative to the total weight of the formulation. In some embodiments, cytisine formulation comprises a glidant in an amount of about 1.5 wt %, relative to the total weight of the formulation.

In some embodiments of the cytisine formulation, the glidant is colloidal silicon dioxide. In some embodiments, the cytisine formulation comprises colloidal silicon dioxide in an amount ranging from about 0.5 wt % to about 5 wt %, relative to a total weight of the formulation. In some embodiments, the cytisine formulation comprises colloidal silicon dioxide in an amount ranging from about 1.0 wt % to about 3.0 wt %, relative to the total weight of the formulation. In some embodiments, the cytisine formulation comprises colloidal silicon dioxide in an amount ranging from about 1.0 wt % to about 2.0 wt %, relative to the total weight of the formulation. In some embodiments, cytisine formulation comprises colloidal silicon dioxide in an amount of about 1.5 wt %, relative to the total weight of the formulation.

In some embodiments, the cytisine formulation comprises a lubricant in an amount ranging from about 0.5 wt % to about 5 wt %, relative to a total weight of the formulation. In some embodiments, the cytisine formulation comprises a lubricant in an amount ranging from about 1.0 wt % to about 3.0 wt %, relative to the total weight of the formulation. In some embodiments, the cytisine formulation comprises a lubricant in an amount ranging from about 1.0 wt % to about 2.0 wt %, relative to the total weight of the formulation. In some embodiments, the cytisine formulation comprises a lubricant in an amount of about 1.5 wt %, relative to the total weight of the formulation.

In some embodiments of the cytisine formulation, the lubricant is magnesium stearate. In some embodiments, the cytisine formulation comprises magnesium stearate in an amount ranging from about 0.5 wt % to about 5 wt %, relative to a total weight of the formulation. In some embodiments, the cytisine formulation comprises magnesium stearate in an amount ranging from about 1.0 wt % to about 3.0 wt %, relative to the total weight of the formulation. In some embodiments, the cytisine formulation comprises magnesium stearate in an amount ranging from about 1.0 wt % to about 2.0 wt %, relative to the total weight of the formulation. In some embodiments, the cytisine formulation comprises magnesium stearate in an amount of about 1.5 wt %, relative to the total weight of the formulation.

As previously described, pharmaceutically acceptable excipients that are present in the cytisine formulation in an amount greater than or equal to about 30 wt %, about 40 wt %, about 50 wt %, or about 60 wt %, relative to a total weight of the formulation, may be pretreated with a nitrite scavenger. Accordingly, in cytisine formulations of the present technology, powdered cellulose and/or calcium sulfate dihydrate may be pretreated with a nitrite scavenger. In some embodiments, when powdered cellulose is pretreated with a nitrite scavenger, the powdered cellulose comprises a nitrite content of less than 5 ppm, less than 4 ppm, less than 3 ppm, less than 2 ppm, or less than 1 ppm, relative to a total weight of the cytisine formulation. Likewise, in embodiments in which calcium sulfate dihydrate is pretreated with a nitrite scavenger, the calcium sulfate dihydrate comprises a nitrite content of less than 5 ppm, less than 4 ppm, less than 3 ppm, less than 2 ppm, or less than 1 ppm, relative to a total weight of the cytisine formulation.

In some embodiments, the cytisine formulation further comprises a buffering agent. Inclusion of a buffering agent in the cytisine formulation may further reduce the amount of NNC and/or may reduce an amount of other product impurities. Addition of a buffering agent to the formulation may also reduce and/or eliminate the formation of other standard impurities, such as N-formylcytisine (NFC) in cytisine formulations wherein the nitrite scavenger is L-cysteine. Non-limiting examples of buffering agents that may be included in cytisine formulations of the present technology include monobasic potassium phosphate (KH₂PO₄), dibasic potassium phosphate (K₂HPO₄), monobasic sodium phosphate (NaH₂PO₄), sodium phosphate dibasic (Na₂HPO₄), ammonium phosphate ((NH₄)₃PO₄), dicalcium phosphate (CaHPO₄), and tricalcium phosphate (Ca₃P₂O₈). In some embodiments, the buffering agent is KH₂PO₄.

In particular, a buffering agent may be added to cytisine formulations of the present technology to adjust the pH of the composition. In some embodiments of the cytisine formulation, the buffering agent is present in an amount of about 1 wt % to about 10 wt %, relative to the total weight of the formulation. In some embodiments of the cytisine formulation, the buffering agent is present in an amount of about 1 wt % to about 5 wt %, relative to the total weight of the formulation. In some embodiments of the cytisine formulation, the buffering agent is present in an amount of about 1.5 wt % to about 5 wt %, relative to the total weight of the formulation. In some embodiments of the cytisine formulation, the buffering agent is present in an amount of about 1 wt % to about 3 wt %, relative to the total weight of the formulation. In some embodiments of the cytisine formulation, the buffering agent is present in an amount of about 1.5 wt % to about 3 wt %, relative to the total weight of the formulation. In some embodiments of the cytisine formulation, the buffering agent is present in an amount of about 1.5 wt % to about 2.5 wt %, relative to the total weight of the formulation. In some embodiments of the cytisine formulation, the buffering agent is present in an amount of about 2.3 wt %, relative to the total weight of the formulation.

In some embodiments, the buffering agent comprises a nitrite content, relative to a total weight of the formulation, of less than about 50 ppm, less than about 25 ppm, less than about 10 ppm, or less than about 5 ppm. In some embodiments, the buffering agent comprises a nitrite content, relative to the total weight of the pharmaceutical composition of about 1 ppm to about 10 ppm. In some embodiments, the buffering agent comprises a nitrite content, relative to the total weight of the pharmaceutical composition of less than 1.2 ppm, less than 1.0 ppm, less than 0.8 ppm, less than 0.6 ppm, less than 0.4 ppm, less than 0.2 ppm, or less than 0.1 ppm.

In some embodiments of the cytisine formulation, the buffering agent is KH₂PO₄, and the KH₂PO₄ is present in an amount of about 1 wt % to about 10 wt %, relative to the total weight of the formulation. In some embodiments of the cytisine formulation, KH₂PO₄ is present in an amount of about 1 wt % to about 5 wt %, relative to the total weight of the formulation. In some embodiments of the cytisine formulation, KH₂PO₄ is present in an amount of about 1.5 wt % to about 5 wt %, relative to the total weight of the formulation. In some embodiments of the cytisine formulation, KH₂PO₄ is present in an amount of about 1 wt % to about 3 wt %, relative to the total weight of the formulation. In some embodiments of the cytisine formulation, KH₂PO₄ is present in an amount of about 1.5 wt % to about 3 wt %, relative to the total weight of the formulation. In some embodiments of the cytisine formulation, KH₂PO₄ is present in an amount of about 1.5 wt % to about 2.5 wt %, relative to the total weight of the formulation. In some embodiments of the cytisine formulation, KH₂PO₄ is present in an amount of about 2.3 wt %, relative to the total weight of the formulation.

In some embodiments, the cytisine formulation has a pH of about 4 to about 9.5. In some embodiments, the cytisine formulation has a pH of about 5.2 to about 8. In some embodiments, the cytisine formulation has a pH of about 7 to about 8. In some embodiments, the cytisine formulation has a pH of about 7.5 to about 7.9. In some embodiments, the cytisine formulation has a pH of 7.6, 7.8, or 7.9. In some embodiments, the cytisine formulation has a pH of about 4 to about 6. In some embodiments, the cytisine formulation has a pH of about 5.2 to about 6. In some embodiments, the cytisine formulation has a pH of 5.3 or 5.7.

To control and/or reduce the amount of N-nitrosamine impurities, such as, e.g., N-nitrosocytisine, the amount of water in the cytisine formulations should be limited. For example, cytisine formulations in accordance with the present technology may comprise less than 7 wt % water, relative to a total weight of the formulation. In some embodiments, the cytisine formulation comprises less than 6.5 wt % water, relative to the total weight of the formulation. In some embodiments, the cytisine formulation comprises less than 5.5 wt % water, relative to the total weight of the formulation. In some embodiments, the cytisine formulation comprises about 3 wt % to about 6.5 wt % water, relative to the total weight of the formulation. In some embodiments, the cytisine formulation comprises about 3.5 wt % to about 6.5 wt % water, relative to the total weight of the formulation. In some embodiments, the cytisine formulation comprises about 4 wt % to about 6.5 wt % water, relative to the total weight of the formulation. In some embodiments, the cytisine formulation comprises about 3 wt % to about 5.5 wt % water, relative to the total weight of the formulation. In some embodiments, the cytisine formulation comprises about 3.5 wt % to about 5.5 wt % water, relative to the total weight of the formulation. In some embodiments, the cytisine formulation comprises about 4 wt % to about 5.5 wt % water, relative to the total weight of the formulation.

In some embodiments, the cytisine formulation comprises less than 3 wt % water, relative to a total weight of the formulation. In some embodiments, the cytisine formulation comprises substantially no water to about 3 wt % water, relative to a total weight of the formulation. In some embodiments, the cytisine formulation comprises substantially no water to about 2 wt % water, relative to a total weight of the formulation. In some embodiments, the cytisine formulation comprises substantially no water to about 1 wt % water, relative to a total weight of the formulation. In some embodiments, the cytisine formulation comprises substantially no water to about 0.5 wt % water, relative to a total weight of the formulation. In some embodiments, the cytisine formulation comprises 0.5 wt % to about 3 wt % water, relative to a total weight of the formulation. In some embodiments, the cytisine formulation comprises 0.5 wt % to about 2 wt % water, relative to a total weight of the formulation. In some embodiments, the cytisine formulation comprises 0.5 wt % to about 1 wt % water, relative to a total weight of the formulation. In some embodiments, the cytisine formulation comprises 1 wt % to about 2 wt % water, relative to a total weight of the formulation. In some embodiments, the cytisine formulation comprises substantially no water.

Cytisine formulations described herein may be formulated as pharmaceutical compositions. In some embodiments, the cytisine formulation is formulated as an oral pharmaceutical composition, i.e., as a composition for oral administration that is orally deliverable. An oral pharmaceutical composition in accordance with the present technology may be formulated as one or more dosage units. The terms “dosage unit” and “unit dosage” may be used interchangeably herein and refer to a portion of a pharmaceutical composition that contains an amount of a therapeutic agent suitable for a single administration to provide a therapeutic effect. Such dosage units may be administered one to a plurality (i.e., 1 to about 10, 1 to 8, 1 to 6, 1 to 4, 1 to 3, or 1 to 2) of times per day, or as many times as needed to elicit a therapeutic response.

When the cytisine formulation is formulated as an oral pharmaceutical composition, the oral pharmaceutical composition may be in any suitable, orally deliverable form including, but not limited to, a pill, a capsule, a powder, a granule, and a film. In some embodiments, the oral pharmaceutical composition in the form of a pill, a capsule, or a powder. In some embodiments, the oral pharmaceutical composition is in the form of a pill. Suitable pill formulations include, but are not limited to, tablets, pellets, caplets, and lozenges. In some embodiments, the pill is in the form of a tablet. The tablet may be prepared in various forms such as, for example, an orally disintegrating tablet, a mucoadhesive tablet, a dispersible tablet, a sublingual tablet, a buccal tablet, a chewable tablet, an effervescent tablet, and a lozenge tablet. In some embodiments, the pill is in the form of a tablet, and the tablet is coated. In some embodiments, the pill is in the form of a tablet, and the tablet is uncoated.

The cytisine formulation may further comprise sweetening, flavoring, or coloring agents.

In some embodiments, the cytisine formulation has a total weight ranging from about 95 mg to about 105 mg. In some embodiments, the cytisine formulation has a total weight ranging from about 95 mg to about 100 mg. In some embodiments, the cytisine formulation has a total weight ranging from about 100 mg to about 105 mg. In some embodiments, the cytisine formulation has a total weight of about 95 mg. In some embodiments, the cytisine formulation has a total weight of about 96 mg. In some embodiments, the cytisine formulation has a total weight of about 97 mg. In some embodiments, the cytisine formulation has a total weight of about 98 mg. In some embodiments, the cytisine formulation has a total weight of about 99 mg. In some embodiments, the cytisine formulation has a total weight of about 100 mg. In some embodiments, the cytisine formulation has a total weight of about 101 mg. In some embodiments, the cytisine formulation has a total weight of about 102 mg. In some embodiments, the cytisine formulation has a total weight of about 103 mg. In some embodiments, the cytisine formulation has a total weight of about 104 mg. In some embodiments, the cytisine formulation has a total weight of about 105 mg.

Cytisine formulations described herein may have an extended shelf-life compared to conventional cytisine formulations. For example, the formulation of the present technology may provide cytisine tablet that have a shelf-life at ambient conditions (i.e., 25° C./60% RH) of at least 18 months, at least 20 months, at least 24 months, at least 28 months, at least 32 months, or at least 36 months. In some embodiments, the cytisine formulation has a shelf-life at ambient conditions of at least about 18 months. In some embodiments, the cytisine formulation has a shelf-life at ambient conditions of at least about 24 months. In some embodiments, the cytisine formulation has a shelf-life at ambient conditions of at least about 36 months.

Cytisine formulations of the present technology may have an extended shelf-life because of the low content of N-nitrosamine impurities of the formulation. For example, after about 18 months of storage at ambient conditions, cytisine formulations described herein may comprise N-nitrosocytisine in an amount of about 4 ng/mg or less, relative to a total weight of cytisine. In some embodiments, cytisine formulations described herein comprise N-nitrosocytisine in an amount of about 4 ng/mg or less, relative to the total weight of cytisine, after about 24 months of storage. In some embodiments, cytisine formulations described herein comprise N-nitrosocytisine in an amount of about 4 ng/mg or less, relative to the total weight of cytisine, after about 36 months of storage. In some embodiments, cytisine formulations described herein comprise N-nitrosocytisine in an amount of about 4.2 ng/mg or less, relative to a total weight of cytisine. In some embodiments, cytisine formulations described herein comprise N-nitrosocytisine in an amount of about 4.2 ng/mg or less, relative to the total weight of cytisine, after about 24 months of storage. In some embodiments, cytisine formulations described herein comprise N-nitrosocytisine in an amount of about 4.2 ng/mg or less, relative to the total weight of cytisine, after about 36 months of storage. In other embodiments, after about 18 months of storage at ambient conditions, cytisine formulations described herein comprise N-nitrosocytisine in an amount of about 45 ng/mg or less, relative to a total weight of cytisine. In some embodiments, cytisine formulations described herein comprise N-nitrosocytisine in an amount of about 45 ng/mg or less, relative to the total weight of cytisine, after about 24 months of storage. In some embodiments, cytisine formulations described herein comprise N-nitrosocytisine in an amount of about 4.2 ng/mg or less, relative to the total weight of cytisine, after about 36 months of storage.

### Dosage Unit

In another aspect, the present technology relates to a dosage unit comprising cytisine and a low level of N-nitrosamine impurities, e.g., NNC and/or short chain alkyl N-nitrosamine impurities such as, for example, NDMA, NDEA, NEMA, NIPEA, NDIPA, NMA, NDPA, NPP, and NDBA. In some embodiments, the dosage unit is in the form of the form of a pill such as, e.g., a tablet, a pellet, a caplet, or a lozenge. In some embodiments, the dosage unit is a tablet. The tablet may be prepared in various forms such as, for example, an orally disintegrating tablet, a mucoadhesive tablet, a dispersible tablet, a sublingual tablet, a buccal tablet, a chewable tablet, and an effervescent tablet.

In some embodiments, the dosage unit is a cytisine tablet. In some embodiments, upon storage of the cytisine tablet at about 60° C., the amount of N-nitrosocytisine is about 3 ng/mg or less after about 30 days. In some embodiments, upon storage of the cytisine tablet at ambient conditions (i.e., 25° C./60% RH), the amount of N-nitrosocytisine is about 4 ng/mg or less after about 18 months. In some embodiments, upon storage of the cytisine tablet at ambient conditions, the amount of N-nitrosocytisine is about 4 ng/mg or less after about 24 months or after about 36 months. In some embodiments, upon storage of the cytisine tablet at ambient conditions (i.e., 25° C./60% RH), the amount of N-nitrosocytisine is about 4.2 ng/mg or less after about 18 months. In some embodiments, upon storage of the cytisine tablet at ambient conditions, the amount of N-nitrosocytisine is about 4.2 ng/mg or less after about 24 months or after about 36 months. In other embodiments, upon storage of the cytisine tablet at ambient conditions, the amount of N-nitrosocytisine is about 45 ng/mg or less after about 18 months. In some embodiments, upon storage of the cytisine tablet at ambient conditions, the amount of N-nitrosocytisine is about 45 ng/mg or less after about 24 months or after about 36 months.

Cytisine may be present in the cytisine tablet in an amount sufficient to achieve the desired treatment effect, e.g., smoking cessation. In some embodiments, cytisine is present in an amount of about 0.5 wt % to about 10 wt %, relative a total weight of the cytisine tablet. In some embodiments, cytisine is present in an amount of about 1 wt % to about 10 wt %, relative the total weight of the cytisine tablet. In some embodiments, cytisine is present in an amount of about 0.5 wt % to about 5 wt %, relative the total weight of the cytisine tablet. In some embodiments, cytisine is present in an amount of about 1 wt % to about 5 wt %, relative the total weight of the cytisine tablet. In some embodiments, cytisine is present in an amount of about 1.5 wt % to about 5 wt %, relative the total weight of the cytisine tablet. In some embodiments, cytisine is present in an amount of about 1 wt % to about 3 wt %, relative the total weight of the cytisine tablet. In some embodiments, cytisine is present in an amount of about 1.5 wt % to about 3 wt %, relative the total weight of the cytisine tablet.

The dosage unit may further comprise a nitrite scavenger. In some embodiments, the nitrite scavenger is one or more amino acids, reducing agents, and antioxidants. Non-limiting examples of nitrite scavengers include amino acids such as homocysteine, L-cysteine, D-cysteine, glycine, alanine, methionine, lysine, taurine, glutathione, cystine, amino sulfamic acids, and amino sulfonic acids; reducing agents such as ascorbic acid; and antioxidants such as vitamin A, vitamin E, propyl gallate, glutathione, and butylated hydroxytoluene (BHT). In some embodiments, the nitrite scavenger is L-cysteine. In some embodiments, when the nitrite scavenger is L-cysteine, the L-cysteine is in the form of a salt, such as, e.g., a hydrochloride. In such embodiments, the L-cysteine may be L-cysteine hydrochloride or L-cysteine hydrochloride anhydrous. Accordingly, in some embodiments, the L-cysteine is L-cysteine hydrochloride. In other embodiments, the L-cysteine is L-cysteine hydrochloride anhydrous. In yet other embodiments, the L-cysteine is free base L-cysteine.

In some embodiments of the dosage unit, the nitrite scavenger is present in the composition in an amount sufficient to control and/or reduce the amount of N-nitrosamine impurities, e.g., NNC in the dosage unit. In some embodiments, the nitrite scavenger is present in an amount of about 1 wt % to about 10 wt %, relative to a total weight of the dosage unit. In some embodiments, the nitrite scavenger is present in an amount of about 1 wt % to about 7 wt %, relative to a total weight of the dosage unit. In some embodiments, the nitrite scavenger is present in an amount of about 3 wt % to about 10 wt %, relative to a total weight of the dosage unit. In some embodiments, the nitrite scavenger is present in an amount of about 3 wt % to about 7 wt %, relative to a total weight of the dosage unit. In some embodiments, the nitrite scavenger is present in an amount of about 1 wt % to about 5 wt %, relative to a total weight of the dosage unit. In some embodiments, the nitrite scavenger is present in an amount of about 3 wt % to about 5 wt %, relative to a total weight of the dosage unit. In some embodiments, the nitrite scavenger is present in an amount of about 5 wt % to about 7 wt %, relative to a total weight of the dosage unit. In some embodiments, the nitrite scavenger is present in an amount of about 5 wt %, relative to a total weight of the dosage unit. In some embodiments, the nitrite scavenger is present in an amount of about 3 wt %, relative to a total weight of the dosage unit. In some embodiments, the nitrite scavenger is present in an amount of about 5 wt %, relative to a total weight of the dosage unit. In some embodiments, the nitrite scavenger is present in an amount of about 7 wt %, relative to a total weight of the dosage unit.

In some embodiments of the dosage unit, the nitrite scavenger is L-cysteine, and L-cysteine is present in the dosage unit in an amount sufficient to control and/or reduce the amount of N-nitrosamine impurities, e.g., NNC in the dosage unit. In some embodiments, L-cysteine is present in an amount of about 1 wt % to about 10 wt %, relative to a total weight of the dosage unit. In some embodiments, L-cysteine is present in an amount of about 1 wt % to about 7 wt %, relative to a total weight of the dosage unit. In some embodiments, L-cysteine is present in an amount of about 3 wt % to about 10 wt %, relative to a total weight of the dosage unit. In some embodiments, L-cysteine is present in an amount of about 3 wt % to about 7 wt %, relative to a total weight of the dosage unit In some embodiments, L-cysteine is present in an amount of about 1 wt % to about 5 wt %, relative to a total weight of the dosage nit. In some embodiments, L-cysteine is present in an amount of about 3 wt % to about 5 wt %, relative to a total weight of the dosage unit. In some embodiments, L-cysteine is present in an amount of about 5 wt % to about 7 wt %, relative to a total weight of the dosage unit. In some embodiments, L-cysteine is present in an amount of about 5 wt %, relative to a total weight of the dosage unit. In some embodiments, L-cysteine is present in an amount of about 3 wt %, relative to a total weight of the dosage unit. In some embodiments, L-cysteine is present in an amount of about 5 wt %, relative to a total weight of the dosage unit. In some embodiments, L-cysteine is present in an amount of about 7 wt %, relative to a total weight of the dosage unit.

The dosage unit may further comprise at least one pharmaceutically acceptable excipient. Excipients that may be included in the dosage unit include, for example, fillers, disintegrants, glidants, lubricants, wetting agents, sweetening agents, flavoring agents, and coloring agents. Such pharmaceutically acceptable excipients may be employed in the compositions to improve processability, palatability, stability, and bioavailability of the composition. Suitable pharmaceutically acceptable excipients that may be included in the dosage unit include, but are not limited to, corn starch, rice starch, potato starch, starch 1500, pregelatinized starch, partially pregelatinized starch, microcrystalline cellulose, microfine cellulose, powdered cellulose, ethyl cellulose, glyceryl behenate, sodium starch glycolate, soy polysaccharide (Emcosoy), calcium sulfate, dicalcium phosphate, dibasic calcium phosphate, tricalcium phosphate, calcium carbonate, calcium stearate, zinc stearate, calcium silicate, magnesium stearate, magnesium lauryl sulfate, magnesium carbonate, magnesium oxide, talc, stearic acid, colloidal silicon dioxide, fumed silica (Cab-O-Sil), dextrates, sucrose, dextrose, crospovidone, sorbitol, mannitol, maltrodextrine, and glucose monohydrate.

In some embodiments, the dosage unit includes one or more pharmaceutically acceptable excipients selected from lubricants, glidants, and fillers. In some embodiments, the one or more pharmaceutically acceptable excipients comprise a lubricant. In some embodiments, the one or more pharmaceutically acceptable excipients comprise a glidant. In some embodiments, the one or more pharmaceutically acceptable excipients comprise one or more fillers. In some embodiments, the dosage unit comprises a lubricant, a glidant, and one or more fillers.

In some embodiments, the dosage unit comprises one or more fillers in an amount ranging from about 70 wt % to about 95 wt %, relative to a total weight of the dosage unit. In some embodiments, the dosage unit comprises one or more fillers in an amount ranging from about 75 wt % to about 92.5 wt %, relative to a total weight of the dosage unit. In some embodiments, the dosage unit comprises one or more fillers in an amount ranging from about 80 wt % to about 90 wt %, relative to a total weight of the dosage unit. In some embodiments, the dosage unit comprises one or more fillers in an amount ranging from about 85 wt % to about 87.5 wt %, relative to a total weight of the dosage unit. In some embodiments, the dosage unit comprises one or more fillers in an amount of about 86.7 wt %, relative to a total weight of the dosage unit.

In some embodiments of the dosage unit, the one or more fillers comprise powdered cellulose. In some embodiments the one or more fillers comprise powdered cellulose in an amount ranging from about 20 wt % to about 35 wt %, relative to a total weight of the dosage unit. In some embodiments, the one or more fillers comprise powdered cellulose in an amount ranging from about 25 wt % to about 30 wt %, relative to the total weight of the dosage unit. In some embodiments, the one or more fillers comprise powdered cellulose in an amount of about 28.5 wt %, relative to the total weight of the dosage unit.

In some embodiments of the dosage unit, the one or more fillers comprise calcium sulfate dihydrate. In some embodiments, the one or more fillers comprise calcium sulfate dihydrate in an amount ranging from about 50 wt % to about 70 wt %, relative to a total weight of the dosage unit. In some embodiments, the one or more fillers comprise calcium sulfate dihydrate in an amount ranging from about 55 wt % to about 65 wt %, relative to the total weight of the dosage unit. In some embodiments, the one or more fillers comprise calcium sulfate dihydrate in an amount ranging from about 55 wt % to about 60 wt %, relative to the total weight of the dosage unit. In some embodiments, the one or more fillers comprise calcium sulfate dihydrate in an amount of about 58.2 wt %, relative to the total weight of the dosage unit.

The one or more fillers may be pretreated with the nitrite scavenger. In some embodiments, the powdered cellulose is pretreated with the nitrite scavenger. In some embodiments, the calcium sulfate dihydrate is pretreated with the nitrite scavenger. In some embodiments, the powdered cellulose and the calcium sulfate dihydrate are pretreated with the nitrite scavenger.

Accordingly, in some embodiments of the dosage unit, the one or more fillers comprise a nitrite content, relative to a total weight of the dosage unit, of less than about 50 ppm, less than about 25 ppm, less than about 10 ppm, or less than about 5 ppm. In some embodiments, the one or more fillers comprise a nitrite content, relative to the total weight of the dosage unit, of equal to or less than about 1.2 ppm, less than about 1.0 ppm, less than about 0.8 ppm, less than about 0.6 pp, less than about 0.4 ppm, less than about 0.2 ppm, or less than about 0.1 ppm.

In some embodiments, the dosage unit comprises a glidant in an amount ranging from about 0.5 wt % to about 5 wt %, relative to a total weight of the dosage unit. In some embodiments, the dosage unit comprises a glidant in an amount ranging from about 1.0 wt % to about 3.0 wt %, relative to the total weight of the dosage unit. In some embodiments, the dosage unit comprises a glidant in an amount ranging from about 1.0 wt % to about 2.0 wt %, relative to the total weight of the dosage unit. In some embodiments, dosage unit comprises a glidant in an amount of about 1.5 wt %, relative to the total weight of the dosage unit.

In some embodiments of the dosage unit, the glidant is colloidal silicon dioxide. In some embodiments, the dosage unit comprises colloidal silicon dioxide in an amount ranging from about 0.5 wt % to about 5 wt %, relative to a total weight of the dosage unit. In some embodiments, the dosage unit comprises colloidal silicon dioxide in an amount ranging from about 1.0 wt % to about 3.0 wt %, relative to the total weight of the dosage unit. In some embodiments, the dosage unit comprises colloidal silicon dioxide in an amount ranging from about 1.0 wt % to about 2.0 wt %, relative to the total weight of the dosage unit. In some embodiments, dosage unit comprises colloidal silicon dioxide in an amount of about 1.5 wt %, relative to the total weight of the dosage unit.

In some embodiments, the dosage unit comprises a lubricant in an amount ranging from about 0.5 wt % to about 5 wt %, relative to a total weight of the dosage unit. In some embodiments, the dosage unit comprises a lubricant in an amount ranging from about 1.0 wt % to about 3.0 wt %, relative to the total weight of the dosage unit. In some embodiments, the dosage unit comprises a lubricant in an amount ranging from about 1.0 wt % to about 2.0 wt %, relative to the total weight of the dosage unit. In some embodiments, the dosage unit comprises a lubricant in an amount of about 1.5 wt %, relative to the total weight of the dosage unit.

In some embodiments of the dosage unit, the lubricant is magnesium stearate. In some embodiments, the dosage unit comprises magnesium stearate in an amount ranging from about 0.5 wt % to about 5 wt %, relative to a total weight of the dosage unit. In some embodiments, the dosage unit comprises magnesium stearate in an amount ranging from about 1.0 wt % to about 3.0 wt %, relative to the total weight of the dosage unit. In some embodiments, the dosage unit comprises magnesium stearate in an amount ranging from about 1.0 wt % to about 2.0 wt %, relative to the total weight of the dosage unit. In some embodiments, the dosage unit comprises magnesium stearate in an amount of about 1.5 wt %, relative to the total weight of the dosage unit.

In some embodiments, the dosage unit comprises a buffering agent to adjust a pH of the dosage unit to a pH ranging from about 4.0 to about 9.5. In some embodiments, the pH of the smoking cessation combination is adjusted to a pH ranging from 4.0 to 9.0. In some embodiments, the pH of dosage unit is adjusted to a pH ranging from 4.0 to 8.5. In some embodiments, the pH of the dosage unit is adjusted to a pH ranging from 4.0 to 8.0. In some embodiments, the pH of the dosage unit is adjusted to a pH ranging from 4.5 to 9.0. In some embodiments, the pH of the dosage unit is adjusted to a pH ranging from 4.5 to 8.5. In some embodiments, the pH of dosage unit is adjusted to a pH ranging from 4.5 to 8.0. In some embodiments, the pH of the dosage unit is adjusted to a pH ranging from 5.0 to 9.0. In some embodiments, the pH of the dosage unit is adjusted to a pH ranging from 5.0 to 8.5. In some embodiments, the pH of the dosage unit is adjusted to a pH ranging from 5.0 to 8.0. In some embodiments, the pH of the dosage unit is adjusted to a pH ranging from 5.3 to 8.0. In some embodiments, the pH of the dosage unit is adjusted to a pH of about 5.2 to about 8. In some embodiments, the pH of the dosage unit is adjusted to a pH of about 7 to about 8. In some embodiments, the pH of the dosage unit is adjusted to a pH of about 7.5 to about 7.9. In some embodiments, the pH of the dosage unit is adjusted to a pH of 7.6, 7.8, or 7.9. In some embodiments, the pH of the dosage unit is adjusted to a pH of about 4 to about 6. In some embodiments, the pH of the dosage unit is adjusted to a pH of about 5.2 to about 6. In some embodiments, the pH of the dosage unit is adjusted to a pH of 5.3 or 5.7.

The buffering agent may be as previously described with reference to the cytisine formulation. In some embodiments, the dosage unit comprises KH₂PO₄.

In some embodiments of the dosage unit, the buffering agent is present in an amount of about 1 wt % to about 10 wt %, relative to the total weight of the dosage unit. In some embodiments, the buffering agent is present in an amount of about 1 wt % to about 5 wt %, relative to the total weight of the dosage unit. In some embodiments, the buffering agent is present in an amount of about 1.5 wt % to about 5 wt %, relative to the total weight of the dosage unit. In some embodiments, the buffering agent is present in an amount of about 1 wt % to about 3 wt %, relative to the total weight of the dosage unit. In some embodiments, the buffering agent is present in an amount of about 1.5 wt % to about 3 wt %, relative to the total weight of the dosage unit. In some embodiments, the buffering agent is present in an amount of about 1.5 wt % to about 2.5 wt %, relative to the total weight of the dosage unit. In some embodiments, the buffering agent is present in an amount of about 2.3 wt %, relative to the total weight of the dosage unit.

In some embodiments of the dosage unit, the buffering agent is KH₂PO₄, and the KH₂PO₄ is present in an amount of about 1 wt % to about 10 wt %, relative to the total weight of the dosage unit. In some embodiments, KH₂PO₄ is present in an amount of about 1 wt % to about 5 wt %, relative to the total weight of the dosage unit. In some embodiments, KH₂PO₄ is present in an amount of about 1.5 wt % to about 5 wt %, relative to the total weight of the dosage unit. In some embodiments, KH₂PO₄ is present in an amount of about 1 wt % to about 3 wt %, relative to the total weight of the dosage unit. In some embodiments, KH₂PO₄ is present in an amount of about 1.5 wt % to about 3 wt %, relative to the total weight of the dosage unit. In some embodiments, KH₂PO₄ is present in an amount of about 1.5 wt % to about 2.5 wt %, relative to the total weight of the dosage unit. In some embodiments, KH₂PO₄ is present in an amount of about 2.3 wt %, relative to the total weight of the dosage unit.

In some embodiments, the dosage unit comprises less than 7 wt % water, relative to a total weight of the dosage unit. In some embodiments, the dosage unit comprises less than 6.5 wt % water, relative to the total weight of the dosage unit. In some embodiments, the dosage unit comprises less than 5.5 wt % water, relative to the total weight of the dosage unit. In some embodiments, the dosage unit comprises about 3 wt % to about 6.5 wt % water, relative to the total weight of the dosage unit. In some embodiments, the dosage unit comprises about 3.5 wt % to about 6.5 wt % water, relative to the total weight of the dosage unit. In some embodiments, the dosage unit comprises about 4 wt % to about 6.5 wt % water, relative to the total weight of the dosage unit. In some embodiments, the dosage unit comprises about 3 wt % to about 5.5 wt % water, relative to the total weight of dosage unit. In some embodiments, the dosage unit comprises about 3.5 wt % to about 5.5 wt % water, relative to the total weight of the dosage unit. In some embodiments, the dosage unit comprises about 4 wt % to about 5.5 wt % water, relative to the total weight of the dosage unit.

In some embodiments, the dosage unit comprises less than 3 wt % water, relative to a total weight of the dosage unit. In some embodiments, the dosage unit comprises substantially no water to about 3 wt % water, relative to a total weight of the dosage unit. In some embodiments, the dosage unit comprises substantially no water to about 2 wt % water, relative to a total weight of the dosage unit. In some embodiments, the dosage unit comprises substantially no water to about 1 wt % water, relative to a total weight of the dosage unit. In some embodiments, the dosage unit comprises substantially no water to about 0.5 wt % water, relative to a total weight of the dosage unit. In some embodiments, the dosage unit comprises 0.5 wt % to about 3 wt % water, relative to a total weight of the dosage unit. In some embodiments, the dosage unit comprises 0.5 wt % to about 2 wt % water, relative to a total weight of the dosage unit. In some embodiments, the dosage unit comprises 0.5 wt % to about 1 wt % water, relative to a total weight of the dosage unit. In some embodiments, the dosage unit comprises 1 wt % to about 2 wt % water, relative to a total weight of the unit. In some embodiments, the dosage unit comprises substantially no water.

In some embodiments, the dosage unit has a total weight ranging from about 95 mg to about 105 mg. In some embodiments, the dosage unit has a total weight ranging from about 95 mg to about 100 mg. In some embodiments, the dosage unit has a total weight ranging from about 100 mg to about 105 mg. In some embodiments, the dosage unit has a total weight of about 95 mg. In some embodiments, the dosage unit has a total weight of about 96 mg. In some embodiments, the dosage unit has a total weight of about 97 mg. In some embodiments, the dosage unit has a total weight of about 98 mg. In some embodiments, the dosage unit has a total weight of about 99 mg. In some embodiments, the dosage unit has a total weight of about 100 mg. In some embodiments, the dosage unit has a total weight of about 101 mg. In some embodiments, the dosage unit has a total weight of about 102 mg. In some embodiments, the dosage unit has a total weight of about 103 mg. In some embodiments, the dosage unit has a total weight of about 104 mg. In some embodiments, the dosage unit has a total weight of about 105 mg.

The referenced patents, patent applications, and scientific literature referred to herein are hereby incorporated by reference in their entirety as if each individual publication, patent, or patent application were specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As can be appreciated from the disclosure above, the present technology has a wide variety of applications. The present technology is further illustrated by the following examples, which are only illustrative and are not intended to limit the definition and scope of the present technology in any way.

### EXAMPLES

### Example 1. Effect of pH and L-Cysteine on N-Nitrosocytisine (NNC) Levels of Cytisine Formulation

The impact of pH in conjunction with L-cysteine on the NNC formation of cytisine formulations was tested as described below.

### Impact of pH

In addition to the standard tablet pH of 9.09 (coated 8.95), two further experimental tablets were made with added dihydrogen potassium phosphate (KH₂PO₄) buffer at pH 5.46 (coated 5.61) and pH 4.42 (coated 4.37). Both coated and uncoated versions of these tablets were stressed for 30 days, under air, nitrogen, and argon at 60° C. NNC was formed in the coated tablets at 3 to 4 times the level of uncoated tablets. The highest levels of NNC were seen for the low pH tablets although the difference was small, particularly for the uncoated tablets. Neither nitrogen or argon blanketing appeared to have a substantial effect on NNC levels, however values under nitrogen or argon were in general lower than air.

### Use of L-Cysteine as an Additive

The addition of 5% L-cysteine to the cytisine formulation resulted in reduced NNC formation (Table 1). After 30 days stressed at 60° C., for uncoated tablets, the NNC levels were about half that of those with no L-cysteine added. The margin of difference was even greater for the coated tablets where the lowest pH (4.40) also showed the highest levels of NNC. Importantly, after 30 days, the NNC levels for uncoated tablets were around 12 ng/tablet, which would comply with a stringent daily acceptable intake limit of less than 40 ng/day NNC (i.e., less than 13.3 ng/tablet). Tablets were manufactured using a low nitrite cellulose excipient and, additionally three batches were manufactured with microcrystalline cellulose (MCC) in conjunction with 5% L-cysteine. MCC had a negative impact on formation of related impurities, mainly due to increased N-formyl cytisine (NFC) formation.

The results that were obtained for experimental tablet batches are presented in Table 1. Each batch prepared was stored at 60° C. for 30 days under air and with nitrogen and argon blanketing. Both uncoated and coated tablets were studied.

TABLE 1
NNC Results for Experimental Tablet Batches at Different pH Values, and with the Addition of L-Cysteine

| | | NNC (ng/tablet) | | | | NNC (ng/tablet) | | | | NNC (ng/tablet) | | | |
|---|---|---|---|---|---|---|---|---|---|---|---|---|---|
| | | Stored under air | | | | Stored under nitrogen | | | | Stored under argon | | | |
| Formulation | | T0 | T10 | T20 | T30 | T0 | T10 | T20 | T30 | T0 | T10 | T20 | T30 |
| Uncoated | Original composition | 4 | 10 | 13 | 27 | 4 | 9 | 11 | 20 | 4 | 9 | 11 | 18 |
| tablets | pH 9.09 | | | | | | | | | | | | |
| | Original composition | 3 | 8 | 12 | 21 | 3 | 8 | 10 | 16 | 3 | 7 | 9 | 17 |
| | pH 5.46 (KH₂PO₄) | | | | | | | | | | | | |
| | Original composition | 1 | 10 | 18 | 24 | 1 | 11 | 22 | 23 | 1 | 9 | 21 | 26 |
| | pH 4.42 (KH₂PO₄) | | | | | | | | | | | | |
| | 5% L-Cysteine pH 7.64 | 1 | 8 | 11 | 12 | 1 | 5 | 9 | 10 | 1 | 6 | 9 | 12 |
| | 5% L-Cysteine pH | 1 | 7 | 10 | 11 | 1 | 5 | 7 | 7 | 1 | 6 | 8 | 9 |
| | 5.99 (KH₂PO₄) | | | | | | | | | | | | |
| | 5% L-Cysteine pH | 1 | 8 | 13 | 12 | 1 | 8 | 13 | 14 | 1 | 8 | 11 | 13 |
| | 4.40 (KH₂PO₄) | | | | | | | | | | | | |
| | 5% L-Cysteine pH | 1 | 5 | 12 | 10 | 1 | 6 | 9 | 11 | 1 | 6 | 11 | 11 |
| | 7.67 MCC | | | | | | | | | | | | |
| | 5% L-Cysteine pH | 1 | 6 | 10 | 9 | 1 | 6 | 9 | 8 | 1 | 4 | 6 | 9 |
| | 5.47 (KH₂PO₄) MCC | | | | | | | | | | | | |
| | 5% L-Cysteine pH | 1 | 5 | 10 | 10 | 1 | 5 | 8 | 13 | 1 | 5 | 8 | 8 |
| | 4.30 (KH₂PO₄) MCC | | | | | | | | | | | | |
| | 5% Propyl gallate pH 7.17 | 6 | 86 | 192 | 303 | 6 | 26 | 48 | 135 | 6 | 47 | 58 | 151 |
| | 5% Propyl gallate pH | 7 | 78 | 125 | 299 | 7 | 22 | 54 | 126 | 7 | 34 | 57 | 160 |
| | 5.55 (KH₂PO₄) | | | | | | | | | | | | |
| Coated | Original composition | 7 | 37 | 50 | 87 | 7 | 38 | 67 | 93 | 7 | 33 | 54 | 72 |
| tablets | pH 8.95 | | | | | | | | | | | | |
| | Original composition | 7 | 37 | 62 | 91 | 7 | 34 | 60 | 85 | 7 | 33 | 64 | 85 |
| | pH 5.61 (KH₂PO₄) | | | | | | | | | | | | |
| | Original composition | 2 | 55 | 78 | 91 | 2 | 51 | 73 | 81 | 2 | 55 | 69 | 76 |
| | pH 4.37 (KH₂PO₄) | | | | | | | | | | | | |
| | 5% L-Cysteine pH 7.63 | 3 | 25 | 30 | 32 | 3 | 20 | 20 | 20 | 3 | 18 | 20 | 20 |
| | 5% L-Cysteine pH | 3 | 18 | 17 | 22 | 3 | 12 | 14 | 14 | 3 | 12 | 14 | 16 |
| | 5.89 (KH₂PO₄) | | | | | | | | | | | | |
| | 5% L-Cysteine pH | 2 | 39 | 46 | 56 | 2 | 28 | 35 | 42 | 2 | 31 | 36 | 39 |
| | 4.40 (KH₂PO₄) | | | | | | | | | | | | |
| | 5% L-Cysteine pH | 2 | 23 | 31 | 29 | 2 | 15 | 18 | 20 | 2 | 15 | 21 | 19 |
| | 7.72 MCC | | | | | | | | | | | | |
| | 5% L-Cysteine pH | 2 | 16 | 23 | 26 | 2 | 12 | 16 | 18 | 2 | 12 | 18 | 19 |
| | 5.43 (KH₂PO₄) MCC | | | | | | | | | | | | |
| | 5% L-Cysteine pH | 2 | 21 | 27 | 30 | 2 | 16 | 17 | 20 | 2 | 15 | 17 | 19 |
| | 4.39 (KH₂PO₄) MCC | | | | | | | | | | | | |

MCC: Microcrystalline cellulose

### Impact of Tablet pH on NNC Formation

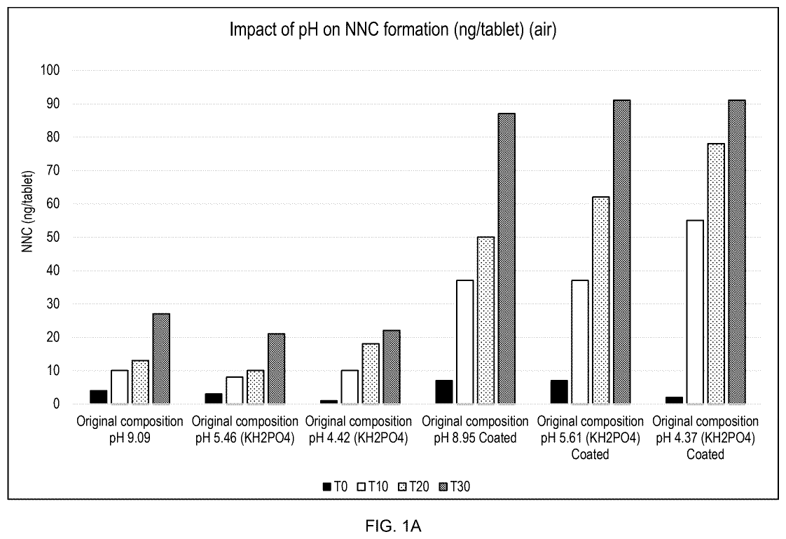

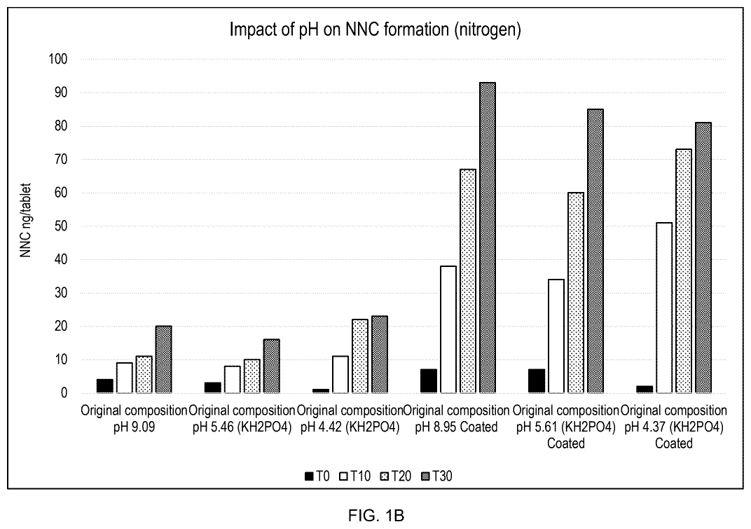

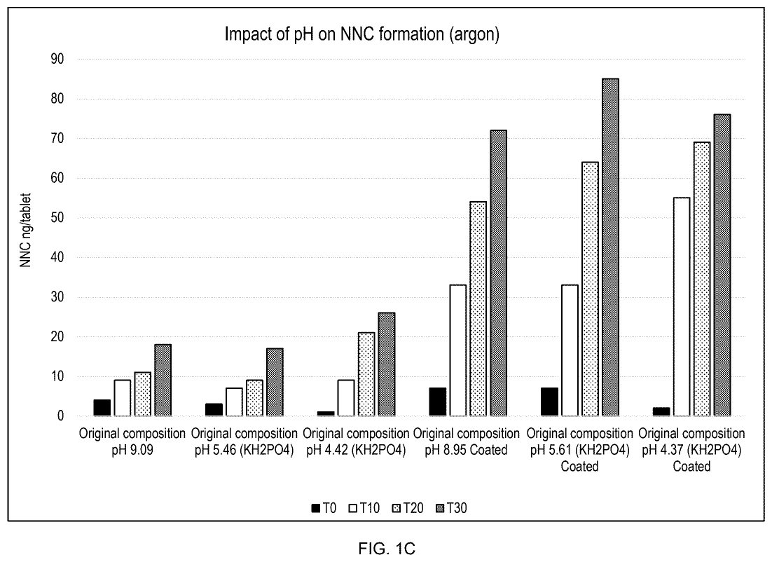

FIGS. 1A-1C show the impact of pH on NNC formation in the original cytisine composition (i.e., cytisine formulation with no additives) stored under air, nitrogen, and argon, respectively. Specifically, the data presented in FIGS. 1A-1C indicates that for the formulation with no additives (other than the dihydrogen potassium phosphate), changes in pH had little effect on NNC formation. Blanketing with either nitrogen or argon did not result in any clear trends, but in general, NNC level were slightly lower than air. Coating of the tablets resulted in a 3-4 fold increase in NNC formation.

### Impact of 5% L-Cysteine on NNC Formation

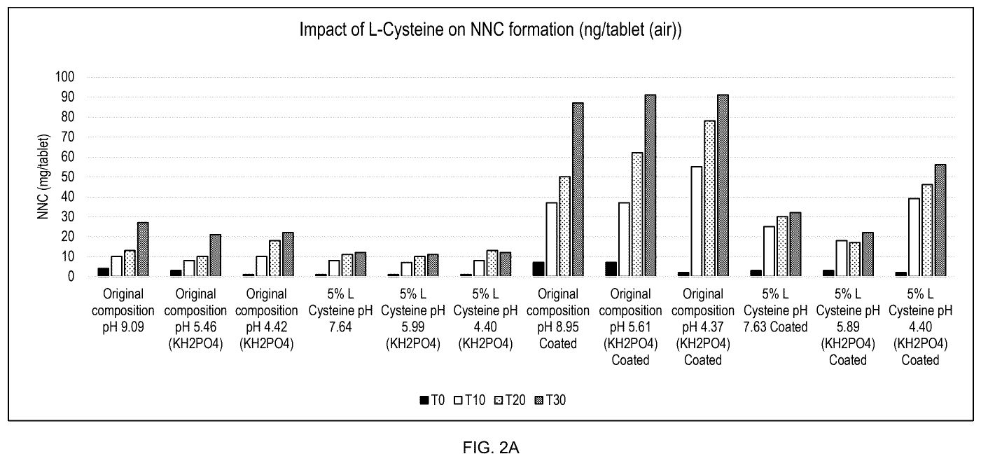

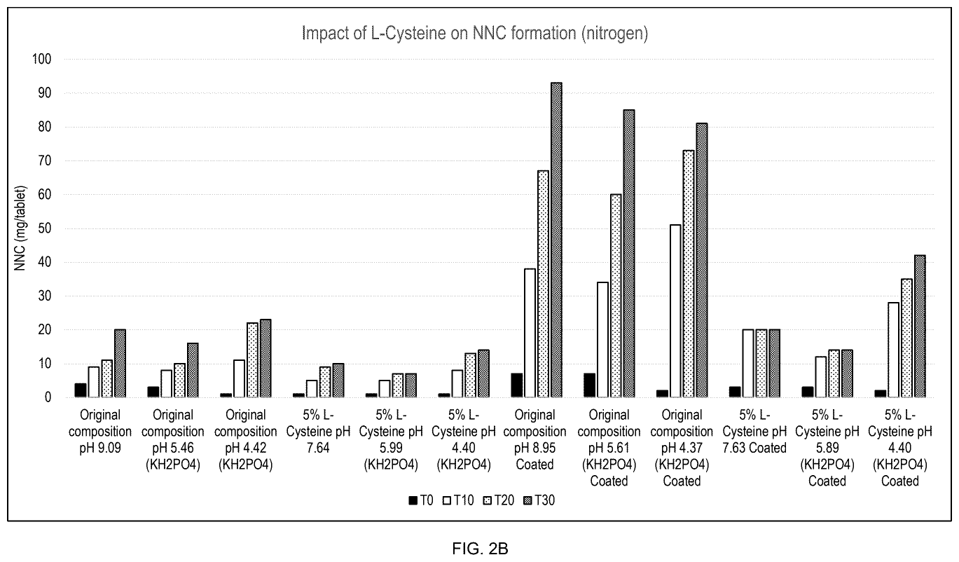

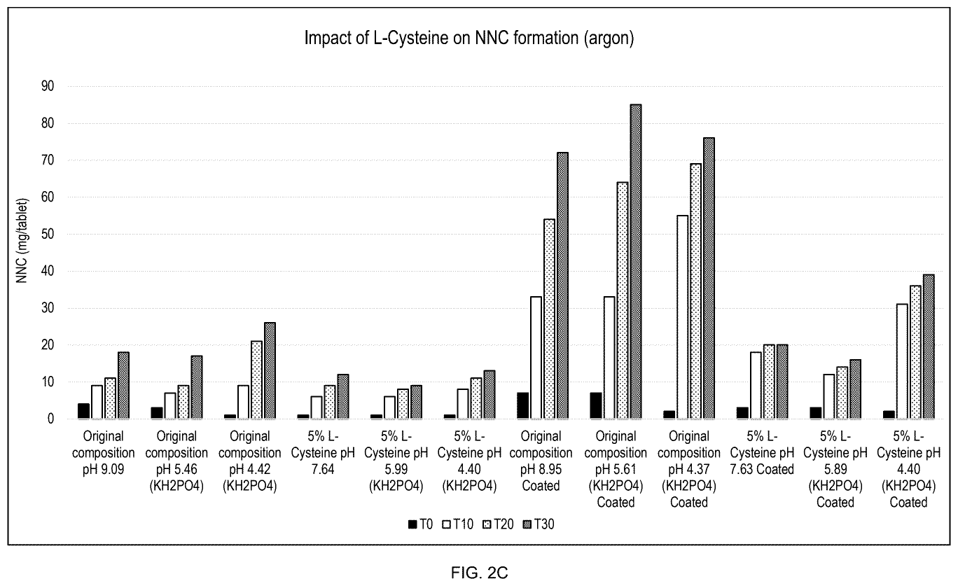

Model tablets were prepared by adding 5% L-cysteine to all three different pH formulations and stressed for 30 days at 60° C. under air, nitrogen, and argon. FIGS. 2A-2C show the impact of the addition of 5% L-cysteine on NNC formation in various cytisine formulations stored under air, nitrogen, and argon, respectively.

FIGS. 2A-2C demonstrate that addition of 5% L-cysteine reduced NNC formation. The effect of L-cysteine was most marked for the coated tablets as the amounts formed were higher, which amplifies the effects. It can also be noted for the coated tablets that the low pH batches had increased NNC levels compared to the mid and high pH formulations. The addition of 5% L-cysteine also had a marked effect on the pH of the control formulation, causing it to fall from about 9 to 7.6.

With the addition of L-cysteine, the rate of NNC formation did not increase as much over time, and in some of the experiments, the level appeared to plateau. This may be an indication that nitrite is being removed from the tablet over time by reaction with L-cysteine. The final levels of NNC after 30 days at 60° C. for uncoated tablets were around 12 ng/tablet.

### Formation of Related Impurities

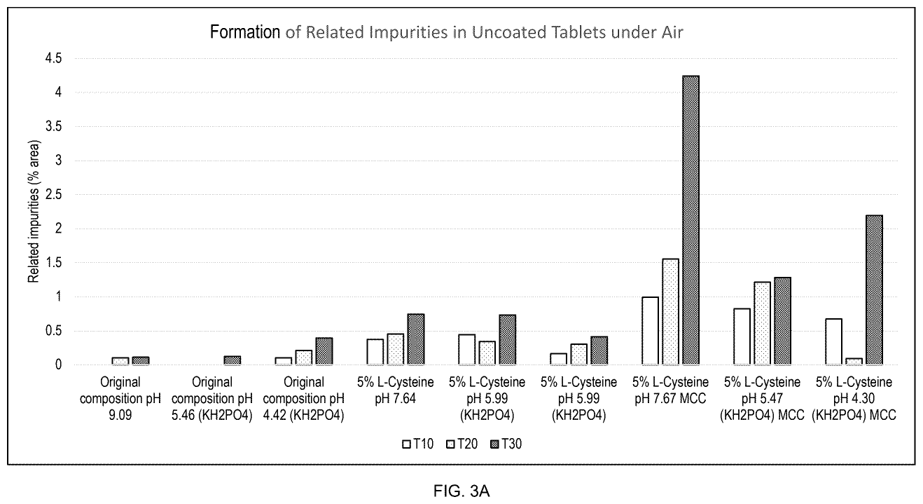

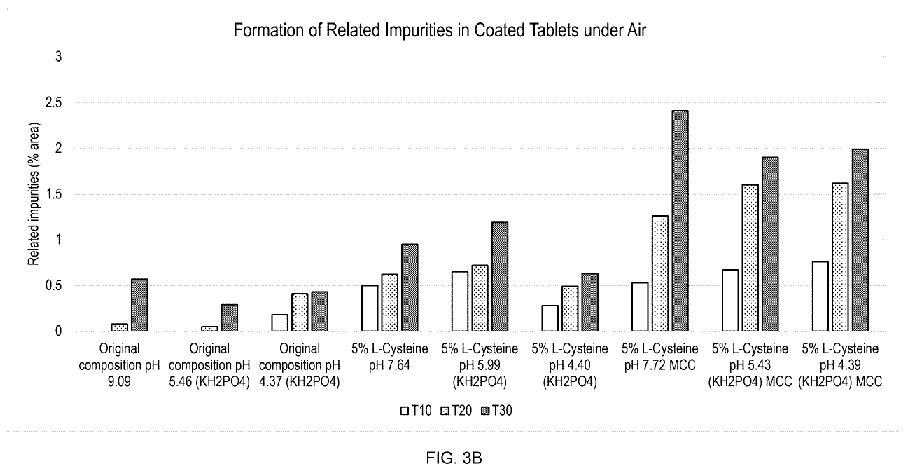

At T0, the total impurity content was <0.05% in all cases. FIGS. 3A and 3B show the general trend observed for total related impurities in uncoated and coated tablets, respectively. FIGS. 3A and 3B show data for tablets stored under air, but the same trends were observed under nitrogen and argon.

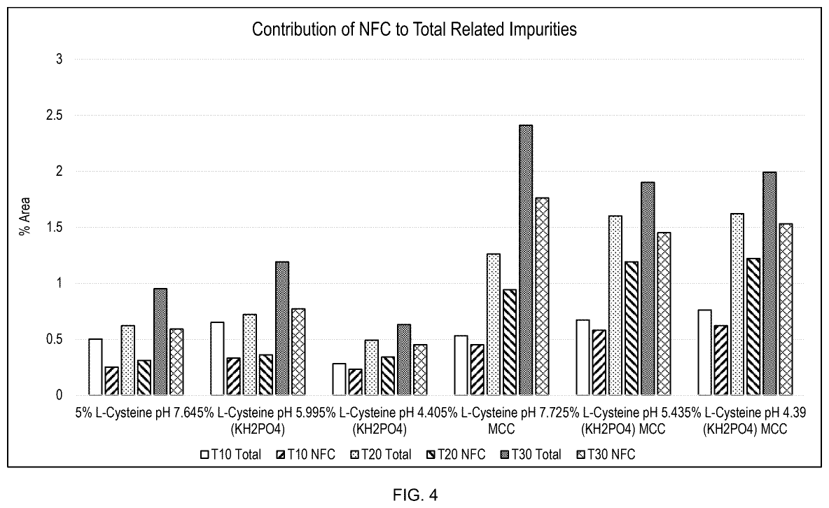

Stressed tablets without L-cysteine resulted in the formation of about 0.5% total impurities. The presence of the coating and the different pH values had little effect on NNC formation. The presence of 5% L-cysteine resulted in an increase in total related impurities. Further analysis of the impurity profile showed that N-formyl cytisine (NFC) made up a significant proportion of the total impurity content. The contribution that NFC makes to the total impurity content is shown in FIG. 4. The data shown in FIG. 4 is for coated tablets stored under air, but the trend is consistent for all batches studied.

In FIG. 4, the blue, grey and purple bars represent the total related impurity content for each time-point, and the orange, yellow and green bars are the contribution due to NFC. In all compositions, NFC was a significant component, particularly for the batches manufactured with MCC. Formation of NFC occurs by reaction with formic acid, so it is likely that this source of MCC contains or forms a higher amount of formic acid than the standard cellulose.

For the tablets made with 5% L-cysteine, the formation of impurities was slightly lower for the low pH tablets.

Additional experiments were conducted to understand the optimum formulation pH and loading of L-cysteine. The results for NNC levels are shown in Tables 2 and 3.

TABLE 2
NNC Results for Additional Experimental Uncoated Tablet Batches

| | NNC (ng/tablet) | | | | NNC (ng/tablet) | | | | NNC (ng/tablet) | | | |
|---|---|---|---|---|---|---|---|---|---|---|---|---|
| | Stored under air | | | | Stored under nitrogen | | | | Stored under argon | | | |
| Formulation | T0 | T10 | T20 | T30 | T0 | T10 | T20 | T30 | T0 | T10 | T20 | T30 |
| Original composition | ND | 10 | 25 | 37 | ND | 8 | 16 | 20 | ND | 10 | 17 | 25 |
| pH 9.05 | | (10) | (13) | (27) | | (9) | (11) | (20) | | (9) | (11) | (18) |
| Original composition | 0.5 | 12 | 23 | 40 | 0.5 | 9 | 17 | 24 | 0.5 | 11 | 19 | 30 |
| pH 7.34 (KH₂PO₄) | | (8) | (12) | (21) | | (8) | (10) | (16) | | (7) | (9) | (17) |
| 1% L-Cysteine pH 8.46 | ND | 8 | 14 | 18 | ND | 11 | 11 | 18 | ND | 10 | 10 | 16 |
| 1% L-Cysteine pH | ND | 9 | 12 | 19 | ND | 8 | 11 | 16 | ND | 9 | 10 | 16 |
| 5.37 (KH₂PO₄) | | | | | | | | | | | | |
| 3% L-Cysteine pH 7.90 | ND | 9 | 14 | 16 | ND | 7 | 9 | 13 | ND | 8 | 9 | 13 |
| 3% L-Cysteine pH | ND | 8 | 14 | 14 | ND | 6 | 8 | 13 | ND | 8 | 9 | 13 |
| 5.38 (KH₂PO₄) | | | | | | | | | | | | |
| 5% L-Cysteine pH 7.83 | ND | 9 | 12 | 14 | ND | 6 | 8 | 14 | ND | 6 | 8 | 11 |
| | | (8) | (11) | (12) | | (5) | (9) | (10) | | (6) | (9) | (12) |
| 5% L-Cysteine pH | ND | 10 | 13 | 13 | ND | 5 | 8 | 13 | ND | 7 | 7 | 11 |
| 5.29 (KH₂PO₄) | | (7) | (10) | (11) | | (5) | (7) | (7) | | (6) | (8) | (9) |
| 7% L-Cysteine pH 7.59 | ND | 8 | 11 | 12 | ND | 5 | 8 | 13 | ND | 6 | 7 | 9 |
| 7% L-Cysteine pH | ND | 8 | 12 | 13 | ND | 5 | 7 | 10 | ND | 6 | 6 | 8 |
| 5.72 (KH₂PO₄) | | | | | | | | | | | | |
| 5% L-Cysteine pH | ND | 5 | 9 | 12 | ND | 4 | 6 | 7 | ND | 7 | 7 | 8 |
| 7.91 (premix heated) | | | | | | | | | | | | |

Results in brackets were those obtained for the same formulation composition in the previous experiment.

TABLE 3
NNC Results for Additional Experimental Coated Tablet Batches

| | NNC (ng/tablet) | | | | NNC (ng/tablet) | | | | NNC (ng/tablet) | | | |
|---|---|---|---|---|---|---|---|---|---|---|---|---|
| | Stored under air | | | | Stored under nitrogen | | | | Stored under argon | | | |
| Formulation | T0 | T10 | T20 | T30 | T0 | T10 | T20 | T30 | T0 | T10 | T20 | T30 |
| Original composition | ND | 64 | 84 | 89 | ND | 58 | 73 | 85 | ND | 58 | 74 | 85 |
| pH 9.07 | | (37) | (50) | (87) | | (38) | (67) | (93) | | (33) | (54) | (72) |
| Original composition | 1 | 48 | 80 | 92 | 1 | 49 | 73 | 84 | 1 | 51 | 73 | 85 |
| pH 7.16 (KH₂PO₄) | | (37) | (62) | (91) | | (34) | (60) | (85) | | (33) | (64) | (85) |
| 1% L-Cysteine pH 8.69 | 1 | 47 | 71 | 78 | 1 | 52 | 61 | 61 | 1 | 49 | 59 | 63 |
| 1% L-Cysteine pH | ND | 43 | 64 | 73 | ND | 40 | 50 | 51 | ND | 39 | 52 | 54 |
| 5.45 (KH₂PO₄) | | | | | | | | | | | | |
| 3% L-Cysteine pH 8.11 | 1 | 33 | 49 | 52 | 1 | 30 | 35 | 34 | 1 | 28 | 36 | 37 |
| 3% L-Cysteine pH | 1 | 32 | 46 | 56 | 1 | 25 | 30 | 34 | 1 | 30 | 32 | 33 |
| 5.44 (KH₂PO₄) | | | | | | | | | | | | |
| 5% L-Cysteine pH 8.23 | 1 | 30 | 28 | 32 | 1 | 19 | 20 | 20 | 1 | 20 | 21 | 28 |
| | | (25) | (30) | (32) | | (20) | (20) | (20) | | (18) | (20) | (20) |
| 5% L-Cysteine pH | 1 | 25 | 30 | 30 | 1 | 19 | 22 | 21 | 1 | 17 | 19 | 18 |
| 5.55 (KH₂PO₄) | | (18) | (17) | (22) | | (12) | (14) | (14) | | (12) | (14) | (16) |
| 7% L-Cysteine pH 7.77 | 2 | 18 | 25 | 26 | 2 | 14 | 16 | 18 | 2 | 15 | 17 | 16 |
| 7% L-Cysteine pH | 2 | 22 | 29 | 28 | 2 | 20 | 16 | 18 | 2 | 20 | 20 | 20 |
| 5.72 (KH₂PO₄) | | | | | | | | | | | | |
| 5% L-Cysteine pH | 1 | 19 | 25 | 23 | 1 | 16 | 20 | 17 | 1 | 18 | 25 | 17 |
| 7.91 (premix heated) | | | | | | | | | | | | |

Results in brackets were those obtained for the same formulation composition in the previous experiment.

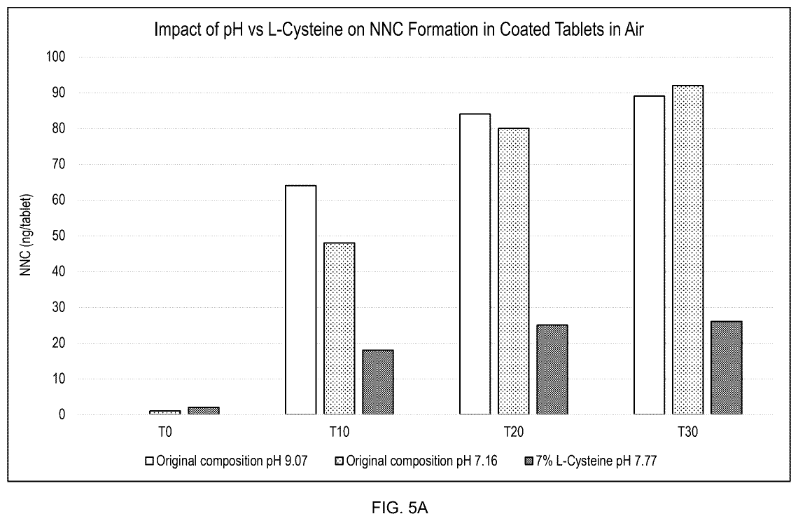

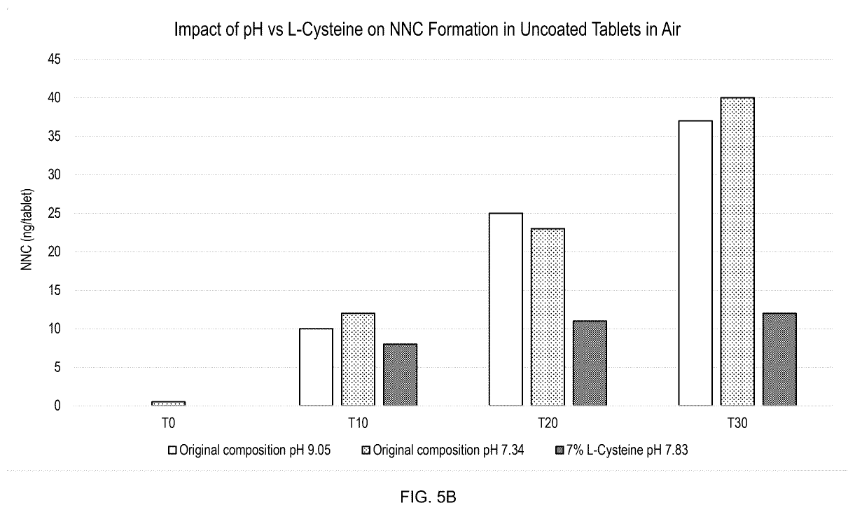

FIGS. 5A and 5B are graphs showing the impact of pH vs L-cysteine on uncoated and coated tablets, respectively. As can be seen in FIG. 5A, there was little difference in the control and pH modified uncoated tablets. However, when 7% L-cysteine was added to the uncoated tablets to give a similar pH range, significant inhibition of NNC formation was observed, confirming that the presence of L-cysteine is required and pH alone is not a significant factor. A similar effect was seen with coated tablets (FIG. 5B).

As L-cysteine provided good inhibition of NNC in the cytisine formulation, a final study was conducted to determine whether there was any trend related to the amount added. L-cysteine was added at different loading levels ranging from 1 wt % to 7% wt. At each loading level, two experimental tablet batches were produced, one with no pH modification and one with addition of potassium dihydrogen phosphate to a pH range between 5 and 5.5.

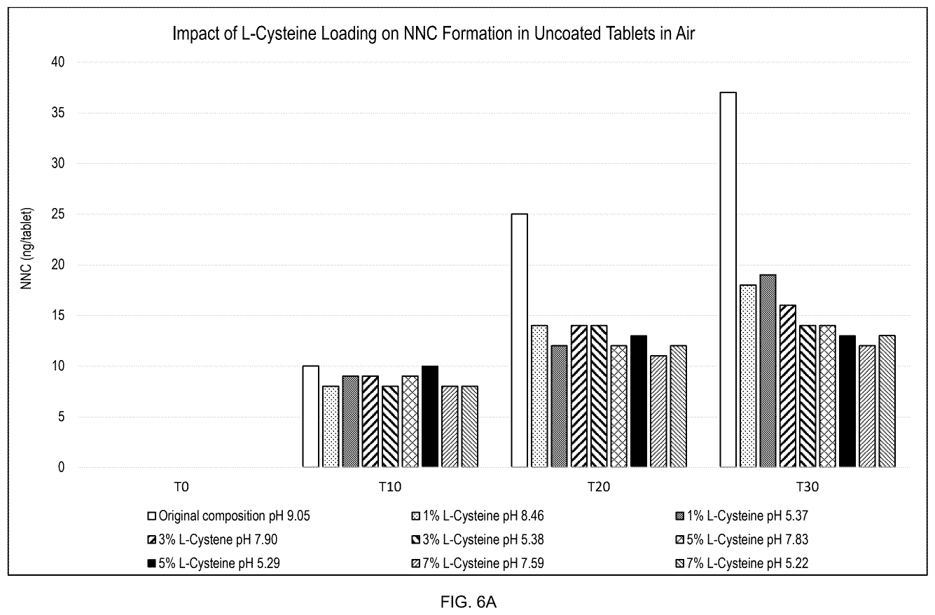

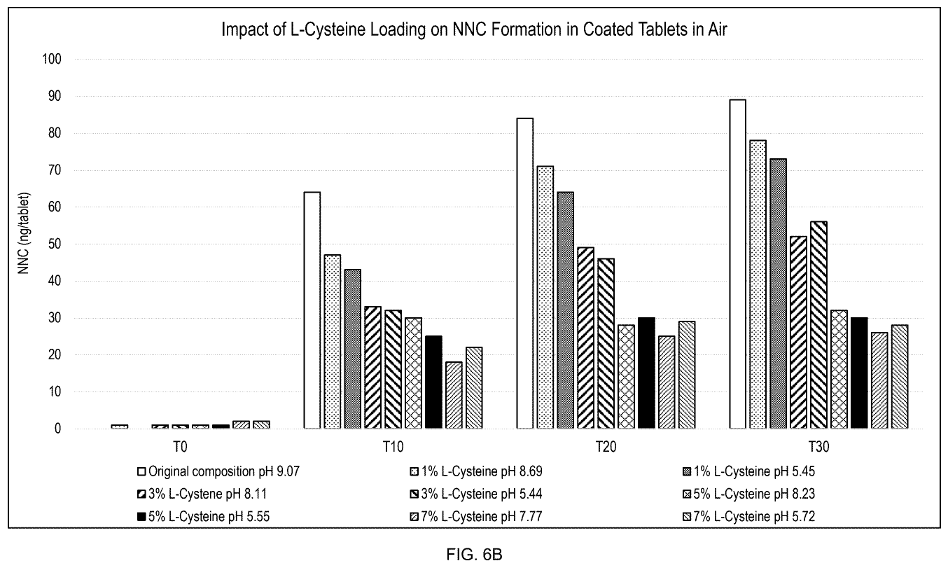

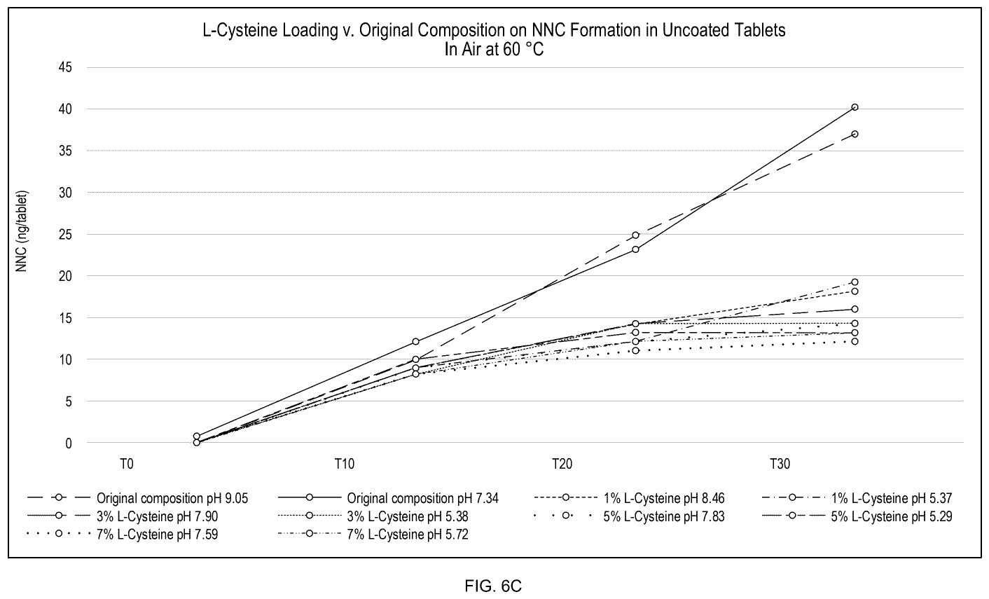

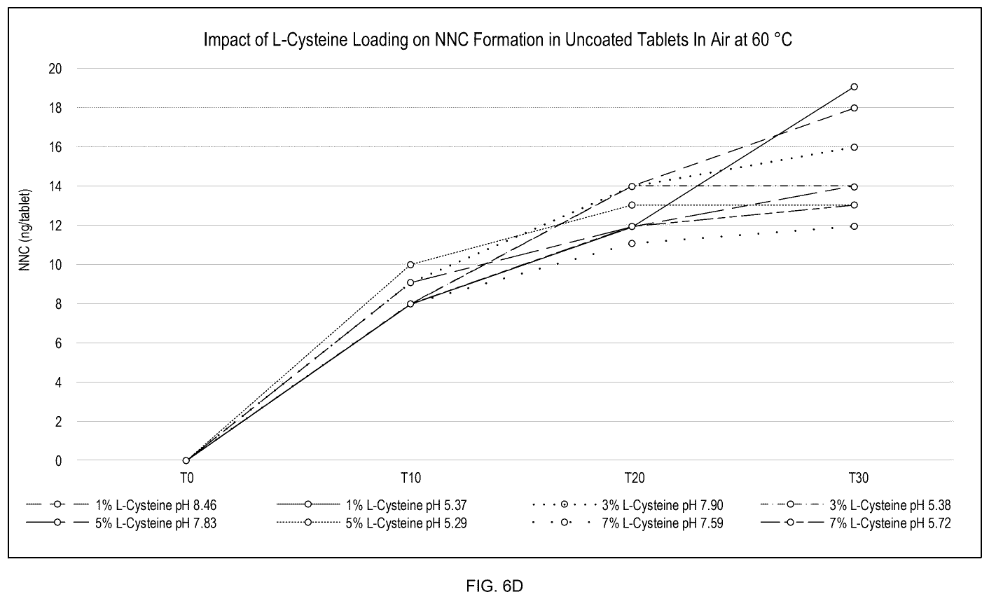

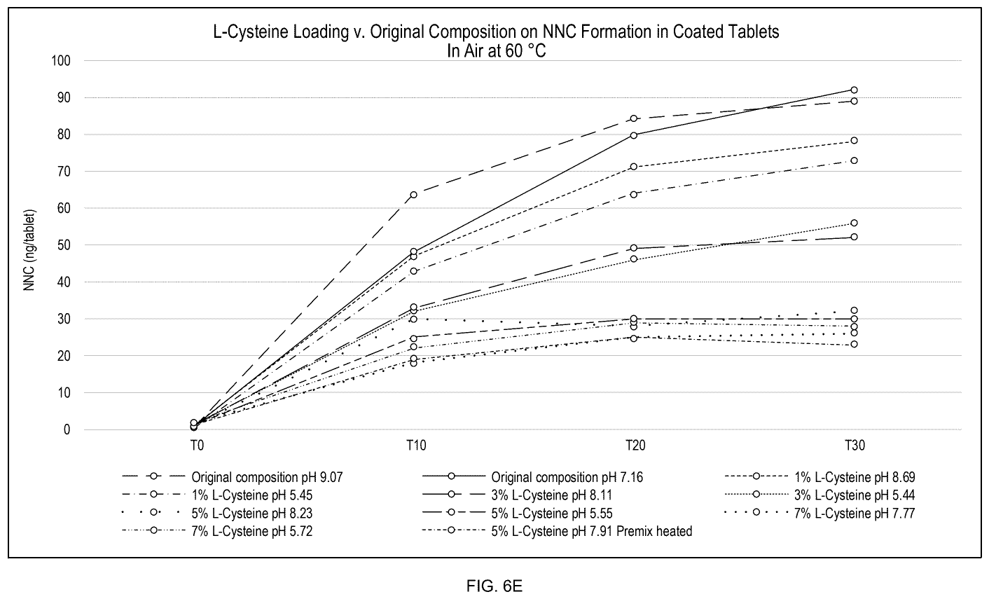

FIGS. 6A and 6B are graphs showing the results of the L-cysteine loading study in uncoated and coated tablets under air. As shown in FIG. 6B, for coated tablets, the increase from 1% to 7% L-cysteine did not have a significant effect. In coated tablets, effects were amplified due to the higher amounts of NNC formed; after 20 and 30 days storage at 60° C., NNC levels were much lower for 5% and 7% L-cysteine loading. Modification of the pH did not have a significant effect on NNC formation. FIGS. 6C-6E are additional graphical representations showing the effect of L-cysteine loading on the NNC formation in uncoated and coated tablets.

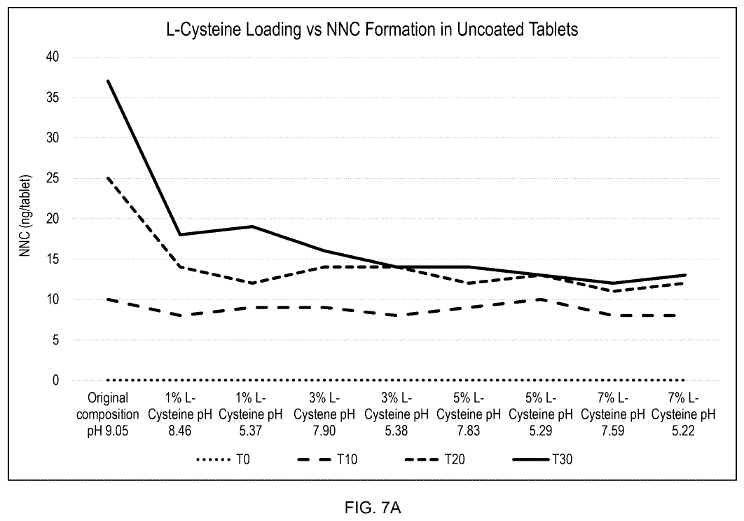

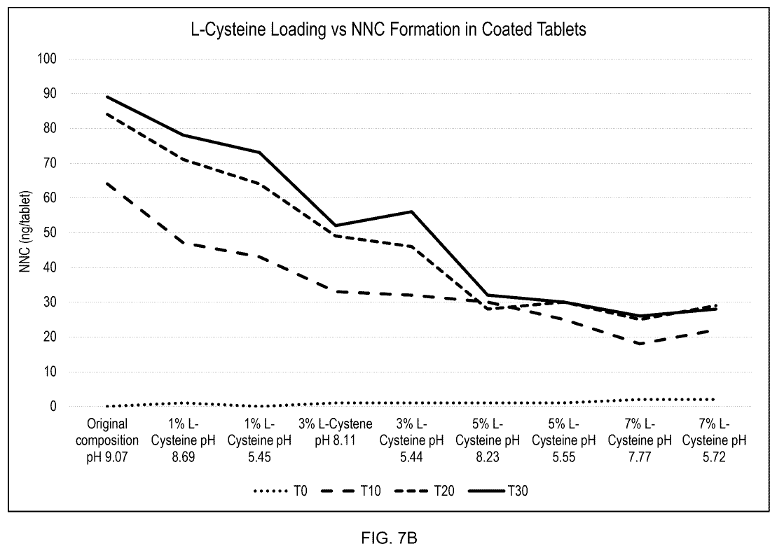

Further graphical illustrations of the effect of L-cysteine loading on uncoated and coated tablets are shown in FIGS. 7A and 7B.

### Example 2. Effect of Pretreating Excipients on NNC Levels of Drug Product

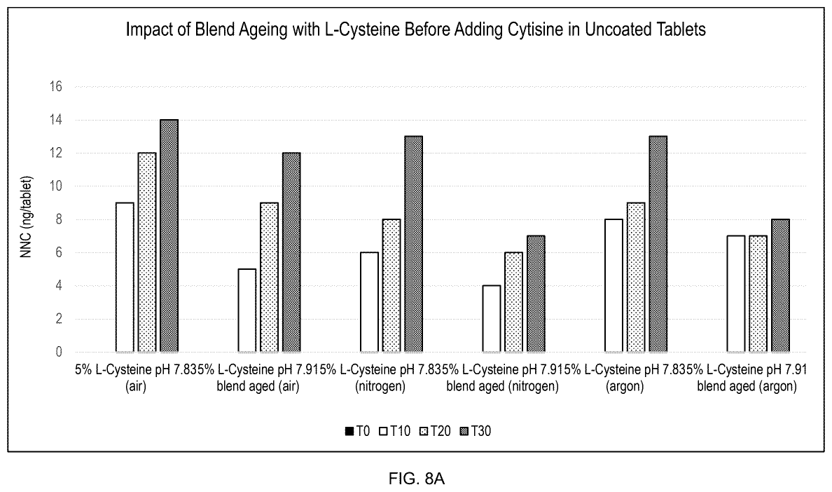

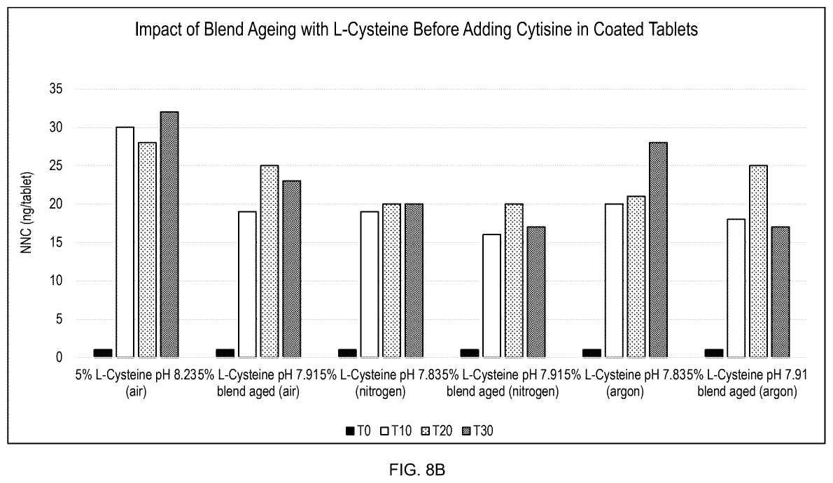

When all the cytisine formulation components and L-cysteine are blended together at the same time, the nitrite scavenging reaction will be in direct competition with the nitrosation of cytisine. Therefore, a study was conducted where the main excipients (cellulose and calcium sulphate) were pre-mixed with 5% L-cysteine and held at 60° C. for 36 h, before continuing with the formulation process where cytisine API was added. The experimental tablets were stressed for 30 days under air, nitrogen and argon. The effect of pretreating the main excipients with the nitrate scavenger in uncoated and coated tablets is shown in the graphs of FIGS. 8A and 8B.

A positive effect when ageing the L-cysteine/cellulose/calcium sulphate was observed, which is best noted by comparing the 30-day bar between the 1^{st} and 2^{nd} (tablets stressed under air), 3^{rd} and 4^{th} (tablets stressed under nitrogen), and 5^{th} and 6^{th} (tablets stressed under argon) entries in the graphs shown in FIGS. 8A and 8B. The most marked effect was seen for the uncoated tablets stored under nitrogen or argon where about a 40% reduction in NNC was observed. The effect is consistent with a partial scavenging of nitrite occurring prior to addition of cytisine.

### Example 3. Cytisine Formulation with Extended Shelf-Life

Based on the foregoing examples, a cytisine formulation having an extended shelf-life has been developed “L-Cysteine Extended Shelf-Life Formulation”. The L-Cysteine Extended Shelf-Life Formulation and the original cytisine formulation are detailed in Table 4.

TABLE 4
L-Cysteine Formulations

| | | |
|---|---|---|
| | | L-Cysteine |
| | Original | Extended |
| | Cytisine | Shelf-Life |
| | Formulation | Formulation |
| Ingredient | [mg/tablet] | [mg/tablet] |

Uncoated Tablet Core

| | | |
|---|---|---|
| Cytisine (equivalent to 100%) | 3 | 3 |
| Powdered cellulose | 28.5 | 28.5 |
| Calcium sulfate, dihydrate | 65.5 | 58.2 |
| Colloidal silicon dioxide | 1.5 | 1.5 |
| Magnesium stearate | 1.5 | 1.5 |
| L-Cysteine | 0 | 5 |
| Potassium dihydrogen phosphate | 0 | 2.3 |
| Mass of one uncoated tablet | 100 | 100 |

Film-Coating

| | | |
|---|---|---|
| Opadry II Brown | 3 | 0 |
| Purified water (evaporates) | N/A | 0 |
| Total mass | 103 mg | 100 mg |

To further control and/or reduce the formation of NNC, the uncoated tablets may be stored under an inert atmosphere and/or certain excipients may be pre-blended/aged.

Various embodiments of the present technology are set forth herein below in paragraphs A to FFF:

Para. A. A cytisine formulation comprising cytisine, a nitrite scavenger, a glidant, a lubricant, a buffering agent, and one or more fillers, wherein, upon storage of the formulation at about 60° C., the amount of N-nitrosocytisine relative to a total weight of cytisine, (c) is 4.2 ng/mg or less after about 30 days.

Para. B. The cytisine formulation of Para. A, wherein cytisine is present in a concentration of about 1 wt % to about 5 wt %, relative to a total weight of the formulation.

Para. C. The cytisine formulation of Para. A or Para. B, wherein cytisine is present in a concentration of about 1.5 wt % to about 3 wt %, relative to a total weight of the formulation.

Para. D. The cytisine formulation of any one of Paras. A-C, wherein the cytisine is pretreated with the nitrite scavenger.

Para. E. The cytisine formulation of any one of Paras. A-D, wherein the nitrite scavenger is selected from the group consisting of an amino acid, a reducing agent, and an antioxidant.

Para. F. The cytisine formulation of Para. E, wherein the amino acid further comprises one or more amino acids selected from the group consisting of homocysteine, L-cysteine, D-cysteine, glycine, alanine, methionine, lysine, taurine, glutathione, and cystine.

Para. G. The cytisine formulation of Para. E, wherein the amino acid further comprises one or more amino sulfamic acids and/or one or more amino sulfonic acids.

Para. H. The cytisine formulation of any one of Paras. E-G, wherein the reducing agent is ascorbic acid.

Para. I. The cytisine formulation of any one of Paras. E-H, wherein the antioxidant further comprises one or more antioxidants selected from the group consisting of vitamin A, vitamin E, propyl gallate, glutathione, and butylated hydroxytoluene (BHT).

Para. J. The cytisine formulation of any one of Paras. A-I, wherein the nitrite scavenger is L cysteine.

Para. K. The cytisine formulation of any one of Paras. A-J, wherein the nitrite scavenger is present in an amount of about 1 wt % to about 10 wt %, relative to a total weight of the formulation.

Para. L. The cytisine formulation of any one of Paras. A-K, wherein nitrite scavenger is present in an amount of about 5 wt % relative to a total weight of the formulation.

Para. M. The cytisine formulation of any one of Paras. A-L, wherein nitrite scavenger is present in an amount of about 7 wt % relative to a total weight of the formulation.

Para. N. The cytisine formulation of any one of Paras. A-M, wherein the glidant is present in a concentration of about 0.5 wt % to about 5 wt %, relative to a total weight of the formulation.

Para. O. The cytisine formulation of any one of Paras. A-N, wherein the glidant is colloidal silicon dioxide.

Para. P. The cytisine formulation of any one of Paras. A-O, wherein the lubricant is present in a concentration of about 0.5 wt % to about 5 wt %, relative to a total weight of the formulation.

Para. Q. The cytisine formulation of any one of Paras. A-P, wherein the lubricant is magnesium stearate.

Para. R. The cytisine formulation of any one of Paras. A-Q, wherein the one or more fillers are present in a concentration of about 70 wt % to about 95 wt %, relative to a total weight of the cytisine formulation.

Para. S. The cytisine formulation of any one of Paras. A-R, wherein the one or more fillers comprise powdered cellulose and calcium sulfate dihydrate.

Para. T. The cytisine formulation of any one of Paras. A-S, wherein the one or more fillers are pretreated with the nitrite scavenger.

Para. U. The cytisine formulation of Para. T, wherein the one or more fillers comprise no more than 1.2 ppm nitrite content relative to a total weight of the formulation.

Para. V. The cytisine formulation of any one of Paras. A-U, wherein the buffering agent is present in a concentration of about 1 wt % to about 5 wt %, relative to a total weight of the formulation.

Para. W. The cytisine formulation of any one of Paras. A-V, wherein the buffering agent is monobasic potassium phosphate (KH₂PO₄).

Para. X. The cytisine formulation of any one of Paras. A-W, wherein the formulation has a pH of about 5.2 to about 8.

Para. Y. The cytisine formulation of any one of Paras. A-X, wherein the formulation has a pH of about 7 to about 8.

Para. Z. The cytisine formulation of any one of Paras. A-Y, wherein the formulation has a pH of about 7.5 to about 7.9.

Para. AA. The cytisine formulation of any one of Paras. A-Z, wherein the formulation has a pH of 7.6, 7.8, or 7.9.

Para. BB. The cytisine formulation of any one of Paras. A-X, wherein the formulation has a pH of about 5.2 to about 6.

Para. CC. The cytisine formulation of any one of Paras. A-X and BB, wherein the formulation has a pH of 5.3 or 5.7.

Para. DD. The cytisine formulation of any one of Paras. A-CC, wherein, upon storage of the formulation at about 60° C., the amount of N-nitrosocytisine, relative to a total weight of cytisine, is about 3 ng/mg or less after 30 days.

Para. EE. The cytisine formulation of any one of Paras. A-DD, wherein, upon storage of the formulation at about 25° C./60% RH, the amount of N-nitrosocytisine, relative to a total weight of cytisine, is about 4 ng/mg or less after 18 months.

Para. FF. The cytisine formulation of any one of Paras. A-EE, wherein, upon storage of the formulation at about 25° C./60% RH, the amount of N-nitrosocytisine, relative to a total weight of cytisine, is about 4 ng/mg or less after 24 months.

Para. GG. The cytisine formulation of any one of Paras. A-FF, wherein the formulation is in the form of an oral pharmaceutical composition.

Para. HH. The cytisine formulation of Para. GG, wherein the oral pharmaceutical composition is in the form of a pill, a capsule, or a powder.

Para. II. The cytisine formulation of Para. HH, wherein the pill is in the form of a tablet, a pellet, a caplet, and a lozenge.

Para. JJ. The cytisine formulation of Para. II, wherein the pill is in the form of a tablet.

Para. KK. The cytisine formulation of Para. HH, wherein the tablet is uncoated.

Para. LL. The cytisine formulation of any one of Paras. A-KK, wherein a total weight of the formulation ranges from about 95 mg to about 105 mg.

Para. MM. A cytisine tablet, comprising 1 wt % to 5 wt % cytisine, 1 wt % to 10 wt % L-cysteine, 0.5 wt % to 5 wt % colloidal silicon dioxide, 0.5 wt % to 5 wt % magnesium stearate, 1 wt % to 5 wt % KH₂PO₄, 20 wt % to 35 wt % powdered cellulose, and 50 wt % to 70 wt % calcium sulfate dihydrate, wherein, upon storage of the tablet at about 60° C., the amount of N-nitrosocytisine relative to a total weight of cytisine, (c) is 4.2 ng/mg or less after about 30 days.

Para. NN. The cytisine tablet of Para. MM, wherein the cytisine is pretreated with the L-cysteine.

Para. OO. The cytisine tablet of Para. MM or Para. NN, wherein the powdered cellulose, the calcium sulfate dihydrate, or both are pretreated with the L-cysteine.

Para. PP. The cytisine tablet of any one of Paras. MM-GO, wherein, upon storage of the tablet at about 25° C./60% RH, the amount of N-nitrosocytisine, relative to a total weight of cytisine, is about 4 ng/mg or less after 18 months.

Para. QQ. The cytisine tablet of any one of Paras. MM-PP, wherein, upon storage of the tablet at about 25° C./60% RH, the amount of N-nitrosocytisine, relative to a total weight of cytisine, is about 4 ng/mg or less after 24 months.

Para. RR. An oral cytisine formulation, comprising: cytisine; an amino acid nitrite scavenger; a glidant; a lubricant; a buffering agent; and one or more fillers, wherein, upon storage of the cytisine formulation at about 25° C./60% RH, the amount of N-nitrosocytisine (NNC) relative to a total weight of cytisine is about 4 ng/mg or less after about 18 months.

Para. SS. The oral cytisine formulation of Para. RR, wherein cytisine is present in a concentration of about 0.5 wt % to about 10 wt %, relative to a total weight of the formulation.

Para. TT. The oral cytisine formulation of Para. RR or SS, wherein the cytisine is pretreated with the amino acid nitrite scavenger.

Para. UU. The oral cytisine formulation of any one of Paras. RR-TT, wherein the amino acid nitrite scavenger comprises one or more amino acids selected from the group consisting of homocysteine, L-cysteine, D-cysteine, glycine, alanine, methionine, lysine, taurine, glutathione, and cystine.

Para. VV. The oral cytisine formulation of any one of Paras. RR-TT, wherein the amino acid nitrite scavenger comprises one or more amino sulfamic acids and/or one or more amino sulfonic acids.

Para. WW. The oral cytisine formulation of any one of Paras. RR-VV, wherein the amino acid nitrite scavenger is L-cysteine.

Para. XX. The oral cytisine formulation of any one of Paras. RR-WW, wherein the amino acid nitrite scavenger is present in an amount of about 1 wt % to about 10 wt %, relative to a total weight of the formulation.

Para. YY. The oral cytisine formulation of any one of Paras. RR-XX, wherein the one or more fillers are pretreated with the amino acid nitrite scavenger.

Para. ZZ. The oral cytisine formulation of Para. YY, wherein the one or more fillers comprise low nitrite content.

Para. AAA. The oral cytisine formulation of any one of Paras. RR-ZZ, wherein the buffering agent is present in a concentration of about 1 wt % to about 5 wt %, relative to a total weight of the formulation.

Para. BBB. The oral cytisine formulation of any one of Paras. RR-AAA, wherein the formulation has a pH of about 4.0 to about 9.5.

Para. CCC. The oral cytisine formulation of any one of Paras. RR-BBB, wherein, upon storage of the formulation at about 25° C./60% RH, the amount of N-nitrosocytisine, relative to a total weight of cytisine, is about 4 ng/mg or less after 24 months.

Para. DDD. The oral cytisine formulation of any one of claims Paras. RR-CCC, wherein the oral formulation is in the form of a pill, a capsule, or a powder.

Para. EEE. The oral cytisine formulation of Para. DDD, wherein the pill is in the form of a pellet, a caplet, and a lozenge.

Para. FFF. An oral dosage unit comprising: cytisine; L-cysteine; a glidant; a lubricant; a buffering agent; and one or more fillers; wherein, upon storage of the composition at about 25° C./60% RH, the amount of N-nitrosocytisine, relative to a total weight of cytisine, is about 4 ng/mg or less after about 18 months.

1. An oral cytisine formulation, comprising:
cytisine;
an amino acid nitrite scavenger;
a glidant;
a lubricant;
a buffering agent; and
one or more fillers,
wherein, upon storage of the cytisine formulation at about 25° C./60% RH, the amount of N-nitrosocytisine (NNC) relative to a total weight of cytisine is about 4 ng/mg or less after about 18 months.

2. The oral cytisine formulation of claim 1, wherein cytisine is present in a concentration of about 0.5 wt % to about 10 wt %, relative to a total weight of the formulation.

3. The oral cytisine formulation of claim 1, wherein the cytisine is pretreated with the amino acid nitrite scavenger.

4. The oral cytisine formulation of claim 1, wherein the amino acid nitrite scavenger comprises one or more amino acids selected from the group consisting of homocysteine, L-cysteine, D-cysteine, glycine, alanine, methionine, lysine, taurine, glutathione, and cystine.

5. The oral cytisine formulation of claim 1, wherein the amino acid nitrite scavenger comprises one or more amino sulfamic acids and/or one or more amino sulfonic acids.

6. The oral cytisine formulation of claim 1, wherein the amino acid nitrite scavenger is L-cysteine.

7. The oral cytisine formulation of claim 1, wherein the amino acid nitrite scavenger is present in an amount of about 1 wt % to about 10 wt %, relative to a total weight of the formulation.

8. The oral cytisine formulation of claim 1, wherein the one or more fillers are pretreated with the amino acid nitrite scavenger.

9. The oral cytisine formulation of claim 8, wherein the one or more fillers comprise low nitrite content.

10. The oral cytisine formulation of claim 1, wherein the buffering agent is present in a concentration of about 1 wt % to about 5 wt %, relative to a total weight of the formulation.

11. The oral cytisine formulation of claim 1, wherein the formulation has a pH of about 4.0 to about 9.5.

12. The oral cytisine formulation of claim 1, wherein, upon storage of the formulation at about 25° C./60% RH, the amount of N-nitrosocytisine, relative to a total weight of cytisine, is about 4 ng/mg or less after 24 months.

13. The oral cytisine formulation of claim 1, wherein the oral formulation is in the form of a pill, a capsule, or a powder.

14. The oral cytisine formulation of claim 13, wherein the pill is in the form of a pellet, a caplet, or a lozenge.

15. An oral dosage unit comprising:
cytisine;
L-cysteine;
a glidant;
a lubricant;
a buffering agent; and
one or more fillers;
wherein, upon storage of the composition at about 25° C./60% RH, the amount of N-nitrosocytisine, relative to a total weight of cytisine, is about 4 ng/mg or less after about 18 months.
